(19) United States
(12) Patent Application Publication
(10) Pub. No.: US 2013/0273098 A1
(43) Pub. Date: Oct. 17, 2013

(54) NOVEL FORMULATIONS WHICH MITIGATE AGITATION-INDUCED AGGREGATION OF IMMUNOGENIC COMPOSITIONS

(71) Applicants: Jeffrey T. Blue, Telford, PA (US); Jayme Cannon, Lexington Park, MD (US); William J. Smith, Harleysville, PA (US); Erin J. Green-Texler, Saratoga, PA (US); Brett Siegfried, Green Lane, PA (US)

(72) Inventors: Jeffrey T. Blue, Telford, PA (US); Jayme Cannon, Lexington Park, MD (US); William J. Smith, Harleysville, PA (US); Erin J. Green-Texler, Saratoga, PA (US); Brett Siegfried, Green Lane, PA (US)

(21) Appl. No.: 13/992,844

(22) PCT Filed: Dec. 5, 2011
(86) PCT No.: PCT/US11/63215

§ 371 Date: Jun. 10, 2013

Related U.S. Application Data
(60) Provisional application No. 61/421,960, filed on Dec. 10, 2010.

(51) Int. Cl. A61K 39/385

(52) U.S. Cl. CPC A61K 39/385
USPC 424/197.11

(57) ABSTRACT

The present invention provides novel formulations which mitigate agitation-induced aggregation of immunogenic compositions particularly those having polysaccharide-protein conjugates. Specifically, the novel formulations comprise a poloxamer within a molecular weight range of 1100 to 17,400 which provides significant advantages over previously used surfactants including polysorbate 80. In one embodiment, the present invention provides a multivalent immunogenic composition having 15 distinct polysaccharide-protein conjugates and a poloxamer. Each conjugate consists of a capsular polysaccharide prepared from a different serotype of Streptococcus pneumoniae (1, 3, 4, 5, 6A, 6B, 7F, 9V, 14, 18C, 19A, 19F, 22F, 23F or 33F) conjugated to a carrier protein, preferably CRM197.

### FIELD OF INVENTION

The present invention provides novel formulations which mitigate agitation-induced aggregation of immunogenic compositions having polysaccharide-protein conjugates. Specifically, the novel formulations comprise a poloxamer surfactant within a molecular weight range of 1100 to 17,400 which provides significant advantages over previously used surfactants including polysorbate 80.

### BACKGROUND OF THE INVENTION

Vaccine formulations must generally be stable and be of uniform consistency to accommodate the need for a long shelf life and the use of multiple dose containers. Vaccines based on proteins, including polysaccharide-protein conjugates, are subject to protein aggregation and precipitation which can result in an effective lower total concentration of the vaccine due to the unavailability of the precipitated protein product. Polysaccharide-protein conjugate vaccines, in particular, appear to have a stronger tendency to aggregate than the carrier protein alone. See Berti et al., 2004, Biophys J 86:3-9.

The choice of formulation for a polysaccharide-protein conjugate vaccine can greatly affect protein aggregation. See Ho et al., 2001, Vaccine 19:716-725. For example, sorbitol has been found to reduce moisture-induced aggregration (see Schwendeman et al., 1995, Proc Natl Acad Sci USA 92:11234-11238) and both Polysorbate 80 and aluminum phosphate were shown to inhibit precipitation of polysaccharide-protein conjugates (see U.S. Patent Application Publication No. 2007/0253984 A1).

There is an ongoing need in the art for additional vaccine formulations which enhance stability and inhibit aggregation/precipitation of immunogenic compositions having polysaccharide-protein conjugates.

### SUMMARY OF THE INVENTION

The present invention relates to novel formulations which inhibit agitation-induced aggregation of immunogenic compositions having one or more polysaccharide-protein conjugates. The formulations of the invention stabilize immunogenic compositions against factors such as silicone oil interactions, shear forces, shipping agitation, thermal stability and the like.

Thus, the invention is directed to formulations comprising (i) a pH buffered saline solution having a pH in the range from 5.0 to 8.0, (ii) a poloxamer having a molecular weight in the range from 1100 to 17,400 and (iii) one or more polysaccharide-protein conjugates.

In certain embodiments, the poloxamer has a molecular weight in the range of 7,500 to 15,000 or 7,500 to 10,000. In certain embodiments, the poloxamer of the formulations is selected from the group consisting of poloxamer 124, poloxamer 188, poloxamer 237, poloxamer 338 and poloxamer 407. In one particular embodiment, the poloxomer is poloxamer 188 or poloxamer 237. In another embodiment, the final concentration of the poloxamer in the formulation is from 0.001% to 5% weight/volume of the formulation. In another embodiment, the final concentration of the poloxamer in the formulation is from 0.025% to 4%, 0.025% to 1%, 0.025% to 0.5%, or 0.025% to 0.15% weight/volume of the formulation. In another embodiment, the final concentration of the poloxamer in the formulation is from 0.05% to 4%, 0.05% to 1%, 0.05% to 0.5%, or 0.05% to 0.15% weight/volume of the formulation. In other embodiments, the final concentration of the poloxamer in the formulation is 0.01%, 0.05%, 0.1%, 0.5%, 1.0% or 5.0% weight/volume of the formulation. In yet another embodiment, the final concentration of poloxamer 188 in the formulation is from 0.05% to 1.0% weight/volume of the formulation or the final concentration of poloxamer 237 in the formulation is from 0.1% to 1.0% weight/volume of the formulation

In certain embodiments, the pH buffered saline solution of the formulations of the invention comprises a buffer having a pH of 5.2 to 8.0, 5.2 to 7.5, or 5.8 to 7.0. In certain embodiments, the buffer is phosphate, succinate, histidine, acetate, citrate, MES, MOPS, TRIS or HEPES. In certain embodiments, the buffer is present at a concentration of 1 mM to 50 mM. In certain embodiments, the buffer is histidine at a final concentration of 5 mM to 50 mM. In one particular embodiment, the final concentration of the histidine buffer is 20 mM.

In certain embodiments, the salt in the pH buffered saline solution comprises magnesium chloride, potassium chloride, sodium chloride or a combination thereof. In one particular embodiment, the salt in the pH buffered saline solution is sodium chloride. In one embodiment, the salt is present at a concentration from 20 mM to 170 mM.

In certain embodiments, the protein of the polysaccharide-protein conjugate formulation is selected from the group consisting of CRM₁₉₇, a tetanus toxoid, a cholera toxoid, a pertussis toxoid, an E. coli heat labile toxoid (LT), a pneumolysin toxoid, pneumococcal surface protein A (PspA), pneumococcal adhesin protein A (PsaA), a C5a peptidase from Streptococcus, Haemophilus influenzae protein D, ovalbumin, keyhole limpet haemocyanin (KLH), bovine serum albumin (BSA) and purified protein derivative of tuberculin (PPD).

In certain embodiments, the polysaccharide-protein conjugate of the formulations comprises one or more pneumococcal polysaccharides. In certain embodiments, the one or more pneumococcal polysaccharides are selected from the group consisting of S. pneumoniae serotype 1 polysaccharide, S. pneumoniae serotype 2 polysaccharide, a S. pneumoniae serotype 3 polysaccharide, a S. pneumoniae serotype 4 polysaccharide, a S. pneumoniae serotype 5 polysaccharide, a S. pneumoniae serotype 6A polysaccharide, a S. pneumoniae serotype 6B polysaccharide, a S. pneumoniae serotype 7F polysaccharide, S. pneumoniae serotype 8 polysaccharide, S. pneumoniae serotype 9N polysaccharide, a S. pneumoniae serotype 9V polysaccharide, S. pneumoniae serotype 10A polysaccharide, S. pneumoniae serotype 11A polysaccharide, S. pneumoniae serotype 12F polysaccharide, a S. pneumoniae serotype 14 polysaccharide, S. pneumoniae serotype 15B polysaccharide, S. pneumoniae serotype 17F polysaccharide, a S. pneumoniae serotype 18C polysaccharide, a S. pneumoniae serotype 19A polysaccharide, a S. pneumoniae serotype 19F polysaccharide, S. pneumoniae serotype 20 polysaccharide, a S. pneumoniae serotype 22F polysaccharide, a S. pneumoniae serotype 23F polysaccharide, and a S. pneumoniae serotype 33F polysaccharide. In one embodiment, the polysaccharide-protein conjugate formulation is a 15-valent pneumococcal conjugate (15vPnC) formulation comprising a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 4 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 6B polysaccharide conjugated to a CRM₁₉₇ polypeptide, a S. pneumoniae serotype 7F polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19A polysaccharide conjugated to a CRM₁₉₇ polypeptide, a S. pneumoniae serotype 19F polysaccharide conjugated to a CRM₁₉₇ polypeptide, a S. pneumoniae serotype 22F polysaccharide conjugated to a CRM₁₉₇ polypeptide, a S. pneumoniae serotype 23F polysaccharide conjugated to a CRM₁₉₇ polypeptide, and a S. pneumoniae serotype 33F polysaccharide conjugated to a CRM₁₉₇ polypeptide.

In certain embodiments, the formulation further comprises an adjuvant. In one embodiment, the adjuvant is an aluminum-based adjuvant, for example, aluminum hydroxide, aluminum phosphate or aluminum sulfate. In one specific embodiment, the aluminum adjuvant is aluminum phosphate. In certain embodiments, the formulation comprises 0.001 mg to 0.250 mg elemental aluminum.

In certain embodiments, the formulation comprises 0.001 mg to 0.250 mg elemental aluminum, preferably, 0.112 mg to 0.130 mg elemental aluminum, 140 to 160 mM sodium chloride and 18 to 22 mM L-histidine buffer. In an exemplary embodiment, the formulation is a single 0.5 mL dose formulated to contain: 1.8 to 2.2 μg of each saccharide, except for 613 at 3.6 to 4.4 μg; about 32 μg CRM₁₉₇ carrier protein; 0.125 mg of elemental aluminum (0.5 mg aluminum phosphate) adjuvant; 150 mM sodium chloride and 20 mM L-histidine buffer.

In certain embodiments, the formulation further comprises a preservative which is m-cresol, phenol, 2-phenoxyethanol, chlorobutanol, benzyl alcohol, or thimerosal.

In certain embodiments, the formulation is contained within a container means selected from the group consisting of a vial, a vial stopper, a vial closure, a glass closure, a rubber closure, a plastic closure, a syringe, including a pre-filled syringe, a syringe stopper, a syringe plunger, a flask, a beaker, a graduated cylinder, a fermentor, a bioreactor, tubing, a pipe, a bag, a jar, an ampoule, a cartridge and a disposable pen. In certain embodiments, the container means is siliconized, preferably baked-on).

### BRIEF DESCRIPTION OF THE DRAWINGS

FIGS. 1A-B: Size Distribution for pH 5.8 Formulation with and without Agitation at 4° C. (A) and 37° C. (B).

FIGS. 2A-B: Size Distribution for pH 5.8 with 0.1% (w/v) Poloxamer 188 Formulation with and without Agitation at 4° C. (A) and 37° C. (B).

FIGS. 3A-B: Size Distribution for pH 7.0 with 0.1% (w/v) Poloxamer 188 Formulation with and without Agitation at 4° C. (A) and (B)

FIGS. 4A-B: Size Distribution for pH 7.0 with 0.1% (w/v) Poloxamer 188, 6% (w/v) Sucrose and 50 mM NaCl Formulation with and without Agitation at 4° C. (A) and 37° C. (B).

FIGS. 5A-D: Size Distritbution for pH 5.8 formulation with and without 0.1% Poloxamer 188 Following ISTA Standard Shipping Study (A) Run 1 from pH 5.8 formulation, (B) Run 2 from pH 5.8 formulation, (C) Run 1 from pH 5.8 formulation with 0.1% Poloxamer 188, (D) Run 2 from 0.1% Poloxamer 188.

FIGS. 6A-C: Size Distribution at pH 5.8, 37° C. with and without agitation under the following conditions: m-cresol in the absence of 0.1% (w/v) Poloxamer 188 (A), m-cresol in the presence of 0.1% (w/v) Poloxamer 188, and phenol in the presence of 0.1% (w/v) Poloxamer 188 (C).

FIG. 7: Size Distribution for pH 5.8 with 0.1% (w/v) Poloxamer 237 Formulation with and without Agitation at 37° C.

### DETAILED DESCRIPTION OF THE INVENTION

The present invention is based, in part, on the discovery that the use of a poloxamer as a surfactant in formulations containing polysaccharide-protein conjugates mitigates agitation induced aggregation and provides unexpectedly superior properties over other surfactants such as polysorbates. The present invention addresses an ongoing need in the art to improve the stability of and inhibit particulate formation (e.g., aggregation, precipitation) of immunogenic compositions such as polysaccharide-protein conjugates.

As described in the Examples, initial studies examined whether the addition of poloxamer 188 at a concentration of 0.1% (w/v) to vaccine formulations could mitigate formation of aggregates upon thermal, mechanical stress, etc. Multiple shake studies as well as simulated shipping studies (ISTA Standard Testing) showed the abiltity of poloxamer 188 to mitigate aggregation following rotational shaking and simulated shipping studies using the current ISTA standard method.

Thus, the invention is directed to a formulation which stabilizes formulation having a polysaccharide-protein conjugate, the formulation comprising a pH buffered saline solution, wherein the buffer has a pH from 5.0 to 8.0, a poloxamer having a molecular weight from 1100 to 17,400 and one or more polysaccharide-protein conjugates. In certain embodiments, the polysaccharide-protein conjugate formulation is comprised in a container means.

As defined herein, the terms “CpG-containing nucleotide,” “CpG-containing oligonucleotide,” “CpG oligonucleotide,” and similar terms refer to a nucleotide molecule of 6-50 nucleotides in length that contains an unmethylated CpG moiety. See, e.g., Wang et al., 2003, Vaccine 21:4297.

As defined herein, the term “polysaccharide” is meant to include any antigenic saccharide element (or antigenic unit) commonly used in the immunologic and bacterial vaccine arts, including, but not limited to, a “saccharide”, an “oligosaccharide”, a “polysaccharide”, a “liposaccharide”, a “lipo-oligosaccharide (LOS)”, a “lipopolysaccharide (LPS)”, a “glycosylate”, a “glycoconjugate” and the like.

As defined herein, a “polysaccharide-protein conjugate”, a “pneumococcal conjugate”, a “7-valent pneumococcal conjugate (7vPnC)”, “13-valent pneumococcal conjugate (13vPnC)”, and “15-valent pneumococcal conjugate (15vPnC)” includes liquid formulations, frozen liquid formulations and solid (e.g., freeze-dried or lyophilized) formulations. The term “PCV 15” also refers to a 15-valent pneumococcal conjugate and is used interchangeably with the term 15vPnC.

As defined herein, the terms “precipitation”, “precipitate” “particulate formation”, “clouding” and “aggregation” may be used interchangeably and are meant to refer to any physical interaction or chemical reaction which results in the “aggregation” of a polysaccharide-protein conjugate. The process of aggregation (e.g., protein aggregation) often influenced by numerous physicochemical stresses, including heat, pressure, pH, agitation, shear forces, freeze-thawing, dehydration, heavy metals, phenolic compounds, silicon oil, denaturants and the like.

As defined herein, a “surfactant” of the present invention is any molecule or compound that lowers the surface tension of an immunogenic composition formulation.

A poloxamer is a nonionic triblock copolymer composed of a central hydrophobic chain of polyoxypropylene (poly(propylene oxide)) flanked by two hydrophilic chains of polyoxyethylene (poly(ethylene oxide)). Poloxamers are also known by the tradename Pluronic®. Because the lengths of the polymer blocks can be customized, many different poloxamers exist that have slightly different properties. For the generic term “poloxamer”, these copolymers are commonly named with the letter “P” (for poloxamer) followed by three digits, the first two digits ×100 give the approximate molecular mass of the polyoxypropylene core, and the last digit ×10 gives the percentage polyoxyethylene content (e.g., P407=Poloxamer with a polyoxypropylene molecular mass of 4,000 g/mol and a 70% polyoxyethylene content). For the Pluronic tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit ×10 gives the percentage polyoxyethylene content (e.g., L61=Pluronic® with a polyoxypropylene molecular mass of 1,800 g/mol and a 10% polyoxyethylene content). See U.S. Pat. No. 3,740,421.

Examples of poloxamers have the general formula:

HO(C₂H₄O)ₐ(C₃H₆O)_{b}(C₂H₄O)ₐH,

wherein a and b blocks have the following values:

| Pluronic ® | Poloxamer | a | b | Molecular Weight |
|---|---|---|---|---|
| L31 | | 2 | 16 | 1100 (average) |
| L35 | | | | 1900 (average) |
| L44NF | 124 | 12 | 20 | 2090 to 2360 |
| L64 | | | | 2900 (average) |
| L81 | | | | 2800 (average) |
| L121 | | | | 4400 (average) |
| P123 | | 20 | 70 | 5750 (average) |
| F68NF | 188 | 80 | 27 | 7680 to 9510 |
| F87NF | 237 | 64 | 37 | 6840 to 8830 |
| F108NF | 338 | 141 | 44 | 12700 to 17400 |
| F127NF | 407 | 101 | 56 | 9840 to 14600 |

Molecular weight units, as used herein, are in Dalton (Da) or g/mol.

In certain embodiments, the final concentration of the poloxamer in the formulation is from 0.001% to 5% weight/volume of the formulation. In another embodiment, the final concentration of the poloxamer in the formulation is from 0.025% to 4%, 0.025% to 1%, 0.025% to 0.5%, or 0.025% to 0.15% weight/volume of the formulation. In another embodiment, the final concentration of the poloxamer in the formulation is from 0.05% to 4%, 0.05% to 1%, 0.05% to 0.5%, or 0.05% to 0.15% weight/volume of the formulation. In other embodiments, the final concentration of the poloxamer in the formulation is 0.01%, 0.05%, 0.1%, 0.5%, 1.0% or 5.0% weight/volume of the formulation. In another embodiment, the final concentration of poloxamer 188 in the formulation is from 0.05% to 1.0% weight/volume of the formulation or the final concentration of poloxamer 237 in the formulation is from 0.1% to 1.0% weight/volume of the formulation.

The surfactant is preferably not conjugated to the carrier protein.

The formulation also contains a pH-buffered saline solution. The buffer may, for example, be selected from the group consisting of TRIS, acetate, glutamate, lactate, maleate, tartrate, phosphate, citrate, carbonate, glycinate, histidine, glycine, succinate, HEPES (4-(2-hydroxyethyl)-1-piperazineethanesulfonic acid), MOPS (3-(N-morpholino)propanesulfonic acid), MES (2-(N-morpholino)ethanesulfonic acid) and triethanolamine buffer. The buffer is capable of buffering a solution to a pH in the range of 4 to 10, 5.2 to 7.5, or 5.8 to 7.0. The buffer may furthermore, for example, be selected from USP compatible buffers for parenteral use, in particular, when the pharmaceutical formulation is for parenteral use. The concentrations of buffer will range from 1 mM to 50 mM or 5 mM to 50 mM.

While the saline solution (i.e., a solution containing NaCl) is preferred, other salts suitable for formulation include but are not limited to, CaCl₂, KCl and MgCl₂. Non-ionic isotonic agents including but not limited to sucrose, trehalose, mannitol, sorbitol and glycerol may be used in lieu of a salt. Suitable salt ranges include, but not are limited to 25 mM to 500 mM or 40 mM to 170 mM

In a preferred embodiment, the vaccine composition is formulated in L-histidine buffer with sodium chloride.

The formulations of the invention may also contain an additional surfactant. Preferred surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (B0), sold under the DOWFAX™ tradename, such as linear ED/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85), and sorbitan monolaurate.

In certain embodiments of the invention, the formulations of the invention are further formulated with an adjuvant. An adjuvant is a substance that enhances the immune response when administered together with an immunogen or antigen. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan. Suitable adjuvants to enhance effectiveness of the composition include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (including, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA;

(3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix (having essentially the same structure as an ISCOM but without the protein (CSL Limited, Parkville, Australia)) described in U.S. Pat. No. 5,254,339;

(4) bacterial lipopolysaccharides, synthetic lipid A analogs such as aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion

(5) synthetic polynucleotides such as oligonucleotides containing CpG motif(s) (U.S. Pat. No. 6,207,646), including those with modified oligonucleotides using any synthetic internucleoside linkages, modified base and/or modified sugar (see, for example, Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110);

(6) cytokines and lymphokines, such as interleukins (e.g., IL-1α, IL-1β, IL-2, IL-4, IL-5, IL-6, IL-7, IL-8, IL-10, IL-12, IL-13, IL-14, IL-15, IL-16, IL-17, IL-18, etc. including mutant forms thereof; U.S. Pat. No. 5,723,127), interferons (e.g., α, β and γ interferon), granulocyte colony stimulating factor (GCSF), granulocyte macrophage colony stimulating factor (GM-CSF), macrophage colony stimulating factor (M-CSF), tumor necrosis factor (TNF), costimulatory molecules B7-1 and B7-2, etc., and chemokines such as MCP-1, MIP-1α, MIP-1β, and RANTES; and

(7) complement, such as a trimer of complement component C3d.

Other adjuvants include Amphigen, Avridine, L121/squalene, D-lactide-polylactide/glycoside, pluronic polyols, muramyl dipeptide, killed Bordetella, Mycobacterium tuberculosis, IC-31 (Intercell AG, Vienna, Austria), described in European Patent Nos. 1,296,713 and 1,326,634, a pertussis toxin (PT), or an E. coli heat-labile toxin (LT), particularly LT-K63, LT-R72, PT-K9/G129; see, e.g., International Patent Publication Nos. WO 93/13302 and WO 92/19265.

In another embodiment, the adjuvant is a mixture of 2, 3, or more of the above adjuvants, e.g., SBAS2 (an oil-in-water emulsion also containing 3-deacylated monophosphoryl lipid A and QS21).

In certain embodiments, the adjuvant is an aluminum salt. The aluminum salt adjuvant may be an alum-precipitated vaccine or an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, alhydrogel, Superfos, Amphogel, aluminum (III) hydroxide, aluminum hydroxyphosphate sulfate (Aluminum Phosphate Adjuvant (APA)), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a target aggregate particle size in the range of 2-8 μm. The product is then diafiltered against physiological saline and steam sterilized.

In certain embodiments, a commercially available Al(OH)₃ (e.g. Alhydrogel or Superfos of Denmark/Accurate Chemical and Scientific Co., Westbury, N.Y.) is used to adsorb proteins in a ratio of 50-200 g protein/mg aluminum hydroxide. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of Ag that is released slowly over a period of 2-3 weeks, be involved in nonspecific activation of macrophages and complement activation, and/or stimulate innate immune mechanism (possibly through stimulation of uric acid). See, e.g., Lambrecht et al., 2009, Curr Opin Immunol 21:23.

A polysaccharide-protein conjugate formulation of the invention can comprise any known polysaccharide and carrier protein. Examples of polysaccharides include pneumococcal polysaccharides, neisserial polysaccharides, and strepotococcus polysaccharides.

In certain embodiments, the one or more pneumococcal polysaccharides are selected from the group consisting of S. pneumoniae serotype 1 polysaccharide, S. pneumoniae serotype 2 polysaccharide, a S. pneumoniae serotype 3 polysaccharide, a S. pneumoniae serotype 4 polysaccharide, a S. pneumoniae serotype 5 polysaccharide, a S. pneumoniae serotype GA polysaccharide, a S. pneumoniae serotype 6B polysaccharide, a S. pneumoniae serotype 7F polysaccharide, S. pneumoniae serotype 8 polysaccharide, S. pneumoniae serotype 9N polysaccharide, a S. pneumoniae serotype 9V polysaccharide, S. pneumoniae serotype 10A polysaccharide, S. pneumoniae serotype 11A polysaccharide, S. pneumoniae serotype 12F polysaccharide, a S. pneumoniae serotype 14 polysaccharide, S. pneumoniae serotype 15B polysaccharide, S. pneumoniae serotype 17F polysaccharide, a S. pneumoniae serotype 18C polysaccharide, a S. pneumoniae serotype 19A polysaccharide, a S. pneumoniae serotype 19F polysaccharide, S. pneumoniae serotype 20 polysaccharide, a S. pneumoniae serotype 22F polysaccharide, a S. pneumoniae serotype 23F polysaccharide, and a S. pneumoniae serotype 33F polysaccharide.

The invention is particularly suitable for multivalent pneumococcal polysaccharide-protein conjugate vaccines containing polysaccharides obtained from multiple serotypes of S. pneumoniae. The 7-valent pneumococcal conjugate vaccine, Prevnar°, contains polysaccharides from serotypes 4, 6B, 9V, 14, 18C, 19F and 23F. U.S. Patent Application Publication No. U.S. 2006/0228380 A1 describes a 13-valent pneumococcal conjugate vaccine including serotypes 1, 3, 4, 5, 6A, 6B, 7F, 9V, 14, 18C, 19A, 19F and 23F. Chinese Patent Application Publication No. CN 101590224 A describes a 14-valent pneumococcal conjugate vaccine including serotypes 1, 2, 4, 5, 6A, 6B, 7F, 9N, 9V, 14, 18C, 19A, 19F and 23F. U.S. Provisional Patent Application No. 61/302,726 describes a 15-valent pneumococcal conjugate vaccine including serotypes 1, 3, 4, 5, 6A, 6B, 7F, 9V, 14, 18C, 19A, 19F, 22F, 23F and 33F.

In certain embodiments, a polysaccharide-protein conjugate formulation is a 7-valent pneumococcal conjugate (7vPnC) formulation comprising a S. pneumoniae serotype 4 polysaccharide conjugated to a CRM₁₉₇ polypeptide, a S. pneumoniae serotype 6B polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide and a S. pneumoniae serotype 23F polysaccharide conjugated to a CRM₁₉₇ polypeptide.

In certain other embodiments, a polysaccharide-protein conjugate formulation is a 13-valent pneumococcal conjugate (13vPnC) formulation comprising a S. pneumoniae serotype 4 polysaccharide conjugated to a CRM₁₉₇ polypeptide, a S. pneumoniae serotype 613 polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19F polysaccharide conjugated to a CRM₁₉₇ polypeptide, a S. pneumoniae serotype 23F polysaccharide conjugated to a CRM₁₉₇ polypeptide, a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 7F polysaccharide conjugated to a CRM₁₉₇ polypeptide and a S. pneumoniae serotype 19A polysaccharide conjugated to a CRM₁₉₇ polypeptide.

In one embodiment, the polysaccharide-protein conjugate formulation is a 15-valent pneumococcal conjugate (15vPnC) formulation comprising a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 4 polysaccharide conjugated to a CRM₁₉₇ polypeptide, a S. pneumoniae serotype 5 polysaccharide conjugated to a CRM197 polypeptide, a S. pneumoniae serotype 6A polysaccharide conjugated to a CRM₁₉₇ polypeptide, a S. pneumoniae serotype 6B polysaccharide conjugated to a CRM₁₉₇ polypeptide, a S. pneumoniae serotype 7F polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19A polysaccharide conjugated to a CRM₁₉₇ polypeptide, a S. pneumoniae serotype 19F polysaccharide conjugated to a CRM₁₉₇ polypeptide, a S. pneumoniae serotype 22F polysaccharide conjugated to a CRM₁₉₇ polypeptide, a S. pneumoniae serotype 23F polysaccharide conjugated to a CRM₁₉₇ polypeptide, and a S. pneumoniae serotype 33F polysaccharide conjugated to a CRM₁₉₇ polypeptide.

Capsular polysaccharides from Steptococeus pneumoniae can be prepared by standard techniques known to those skilled in the art. For example, polysaccharides can be isolated from bacteria and may be sized to some degree by known methods (see, e.g., European Patent Nos. EP497524 and EP497525) and preferably by microfluidisation. Polysaccharides can be sized in order to reduce viscosity in polysaccharide samples and/or to improve filterability for conjugated products.

In one embodiment, each pneumococcal polysaccharide serotype is grown in a soy-based medium. The individual polysaccharides are then purified through standard steps including centrifugation, precipitation, and ultra-filtration. See, e.g., U.S. Patent Application Publication No. 2008/0286838 and U.S. Pat. No. 5,847,112.

Carrier proteins are preferably proteins that are non-toxic and non-reactogenic and obtainable in sufficient amount and purity. A carrier protein can be conjugated or joined with a S. pneumoniae polysaccharide to enhance immunogenicity of the polysaccharide. Carrier proteins should be amenable to standard conjugation procedures. In a particular embodiment of the present invention, CRM₁₉₇ is used as the carrier protein. In one embodiment, each capsular polysaccharide is conjugated to the same carrier protein (each capsular polysaccharide molecule being conjugated to a single carrier protein). In another embodiment, the capsular polysaccharides are conjugated to two or more carrier proteins (each capsular polysaccharide molecule being conjugated to a single carrier protein). In such an embodiment, each capsular polysaccharide of the same serotype is typically conjugated to the same carrier protein.

CRM₁₉₇ is a non-toxic variant (i.e., toxoid) of diphtheria toxin. In one embodiment, it is isolated from cultures of Corynebacterium diphtheria strain C7 (β197) grown in casamino acids and yeast extract-based medium. In another embodiment, CRM₁₉₇ is prepared recombinantly in accordance with the methods described in U.S. Pat. No. 5,614,382. Typically, CRM₁₉₇ is purified through a combination of ultra-filtration, ammonium sulfate precipitation, and ion-exchange chromatography. In some embodiments, CRM₁₉₇ is prepared in Pseudomonas fluorescens using Pfenex Expression Technology™ (Pfenex Inc., San Diego, Calif.).

Other suitable carrier proteins include additional inactivated bacterial toxins such as DT (Diphtheria toxoid), TT (tetanus toxid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal adhesin protein (PsaA), C5a peptidase from Group A or Group B streptococcus (for example, an enzymatically inactive streptococcal C5a peptidase (SCP) such as one or more of the SCP variants described in U.S. Pat. No. 6,951,653, U.S. Pat. No. 6,355,255 and U.S. Pat. No. 6,270,775), or Haemophilus influenzae protein D, pneumococcal pneumolysis (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Other DT mutants can be used, such as CRM₁₇₆, CRM₂₂₃, CRM₄₅ (Uchida et al., 1973, J Biol Chem 218:3838-3844); CRM₉, CRM₄₅, CRM₁₀₂, CRM₁₀₃ and CRM₁₀₇ and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711.

The purified polysaccharides are then chemically activated (e.g., via reductive amination) to make the saccharides capable of reacting with the carrier protein. Once activated, each capsular polysaccharide is separately conjugated by known coupling techniques to a carrier protein (e.g., CRM₁₉₇) to form a glycoconjugate (or alternatively, each capsular polysaccharide is conjugated to the same carrier protein) and formulated into a single dosage formulation.

In one embodiment, the chemical activation of the polysaccharides and subsequent conjugation to the carrier protein are achieved by means described in U.S. Pat. Nos. 4,365,170, 4,673,574 and 4,902,506. Briefly, that chemistry entails the activation of pneumococcal polysaccharide by reaction with any oxidizing agent which oxidizes a terminal hydroxyl group to an aldehyde, such as periodate (including sodium periodate, potassium periodate, or periodic acid). The reaction leads to a random oxidative cleavage of vicinal hydroxyl groups of the carbohydrates with the formation of reactive aldehyde groups.

In one embodiment, coupling to the protein carrier (e.g., CRM₁₉₇) can be by reductive amination via direct amination to the lysyl groups of the protein. For example, conjugation is carried out by reacting a mixture of the activated polysaccharide and carrier protein with a reducing agent such as sodium cyanoborohydride. Unreacted aldehydes are then capped with the addition of a strong reducing agent, such as sodium borohydride.

In another embodiment, the conjugation method may rely on activation of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide [e.g. ethyl iodoacetimide HCl] or N-succinimidyl bromoacetate or STAB, or SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g. EDAC or EDC) chemistry via a carboxyl group on the protein carrier. Such conjugates are described in International Patent Application Publication Nos. WO 93/15760, WO 95/08348 and WO 96/29094; and Chu et al., 1983, Infect. Immunity 40:245-256.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (See Bethell et al., 1979, J. Biol. Chem. 254:2572-4; Ream et al., 1981, J. Chromatogr. 218:509-18) followed by reaction of with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In one embodiment, prior to formulation, each pneumococcal capsular polysaccharide antigen is individually purified from S. pneumoniae, activated to form reactive aldehydes, and then covalently conjugated using reductive amination to the carrier protein CRM₁₉₇.

After conjugation of the capsular polysaccharide to the carrier protein, the polysaccharide-protein conjugates are purified (enriched with respect to the amount of polysaccharide-protein conjugate) by one or more of a variety of techniques. Examples of these techniques are well known to the skilled artisan and include concentration/diafiltration operations, ultrafiltration, precipitation/elution, column chromatography, and depth filtration. See, e.g., U.S. Pat. No. 6,146,902.

After the individual glycoconjugates are purified, they are compounded to formulate the immunogenic composition of the present invention. These pneumococcal conjugates are prepared by separate processes and bulk formulated into a single dosage formulation. Formulation of the polysaccharide-protein conjugates of the present invention can be accomplished using art-recognized methods. For instance, the 15 individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The amount of conjugate in each vaccine dose is selected as an amount that induces an immunoprotective response without significant, adverse effects. Such amount can vary depending upon the pneumococcal serotype. Generally, each dose will comprise 0.1 to 100 μg of each polysaccharide, particularly 0.1 to 10 mg, and more particularly 1 to 5 μg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg.

In one embodiment, the dose of the aluminum salt is 10, 15, 20, 25, 30, 50, 70, 100, 125, 150, 200, 300, 500, or 700 μg, or 1, 1.2, 1.5, 2, 3, 5 mg or more. In yet another embodiment, the dose of alum salt described above is per μg of recombinant protein.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

In a particular embodiment of the present invention, the PCV-15 vaccine is a sterile liquid formulation of pneumococcal capsular polysaccharides of serotypes 1, 3, 4, 5, 6A, 6B, 7F, 9V, 14, 18C, 19A, 19F, 22F, 23F and 33F individually conjugated to CRM₁₉₇. Each 0.5 mL dose is formulated to contain: 2 μg of each saccharide, except for 6B at 4 μg; about 32 μg CRM₁₉₇ carrier protein (e.g., 32 μg±5 μg, ±3 μg, ±2 μg, or ±1 μg); 0.125 mg of elemental aluminum (0.5 mg aluminum phosphate) adjuvant; and sodium chloride and L-histidine buffer. The sodium chloride concentration is about 150 mM (e.g., 150 mM±25 mM, ±20 mM, ±15 mM, ±10 mM, or ±5 mM) and about 20 mM (e.g, 20 mM±5 mM, ±2.5 mM, ±2 mM, ±1 mM, or ±0.5 mM) L-histidine buffer.

The polysaccharide-protein conjugates are typically combined into a blend of the multiple serotypes in the pH-buffered saline, then combined with adjuvant (which may be in saline). The poloxamer may be added at any stage in the process.

In one embodiment, the process consists of combining blend of 15 serotypes in histidine, saline, and poloxamer, then combining this blended material with APA and saline.

In a specific embodiment, the formulation consists of histidine (20 mM), saline (150 mM) and poloxamer 188 (0.1% w/v) at a pH of 5.8 with 250 ug/mL of APA (Aluminum Phosphate Adjuvant). Current efforts have examined pH range from 5.8-7.0 and shown that the formulation listed mitigates agitation-induced aggregation. Range finding for poloxamer 188 is currently underway examining range from 0.025 to 0.15%.

The compositions of this invention may also include one or more proteins from S. pneumoniae. Examples of S. pneumoniae proteins suitable for inclusion include those identified in International Patent Application Publication Nos. WO 02/083855 and WO 02/053761.

The formulations described above may also comprise one or more additional pharmaceutically acceptable diluents, excipients or a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with administration to humans or other vertebrate hosts. The appropriate carrier is evident to those skilled in the art and will depend in large part upon the route of administration.

Pharmaceutically acceptable carriers for liquid formulations are aqueous or non-aqueous solutions, suspensions, emulsions or oils. Examples of nonaqueous solvents are propylene glycol, polyethylene glycol, and injectable organic esters such as ethyl oleate. Aqueous carriers include (in addition to water) alcoholic/aqueous solutions, emulsions or suspensions. Examples of oils are those of animal, vegetable, or synthetic origin, for example, peanut oil, soybean oil, olive oil, sunflower oil, fish-liver oil, another marine oil, or a lipid from milk or eggs.

Excipients that may be present in the immunogenic composition formulation include but are not limited to preservatives, chemical stabilizers and suspending or dispersing agents. Typically, stabilizers, preservatives and the like are optimized to determine the best formulation for efficacy in the targeted recipient (e.g., a human subject). Examples of preservatives include m-cresol, 2-phenoxyethanol, benzyl alcohol, thimerosal, chlorobutanol, potassium sorbate, sorbic acid, sulfur dioxide, propyl gallate, the parabens, ethyl vanillin, glycerin, phenol, and parachlorophenol. Examples of stabilizing ingredients include casamino acids, sucrose, gelatin, phenol red, N-Z amine, monopotassium diphosphate, lactose, lactalbumin hydrolysate, and dried milk.

In certain embodiments, an immunogenic composition formulation is prepared for administration to human subjects in the form of, for example, liquids, powders, aerosols, tablets, capsules, enteric-coated tablets or capsules, or suppositories. Thus, the immunogenic composition formulations may also include, but are not limited to, suspensions, solutions, emulsions in oily or aqueous vehicles, pastes, and implantable sustained-release or biodegradable formulations.

In another embodiment, formulations of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

In certain embodiments, the formulations are single dose vials, multi-dose vials or pre-filled syringes.

The immunogenic compositions of the present invention are not limited by the selection of the conventional, physiologically acceptable carriers, diluents and excipients such as solvents, buffers, adjuvants, or other ingredients useful in pharmaceutical preparations of the types described above. The preparation of these pharmaceutically acceptable compositions, from the above-described components, having appropriate pH, isotonicity, stability and other conventional characteristics is within the skill of the art.

In certain embodiments, the invention is directed to formulations of immunogenic compositions comprised in a container means. As defined herein, a “container means” of the present invention includes any composition of matter which is used to “contain”, “hold”, “mix”, “blend”, “dispense”, “inject”, “transfer”, “nebulize”, etc. an immunogenic composition during research, processing, development, formulation, manufacture, storage and/or administration. For example, a container means of the present invention includes, but is not limited to, general laboratory glassware, flasks, beakers, graduated cylinders, fermentors, bioreactors, tubings, pipes, bags, jars, vials, vial closures (e.g., a rubber stopper, a screw on cap), ampoules, syringes, syringe stoppers, syringe plungers, rubber closures, plastic closures, glass closures, and the like. A container means of the present invention is not limited by material of manufacture, and includes materials such as glass, metals (e.g., steel, stainless steel, aluminum, etc.) and polymers (e.g., thermoplastics, elastomers, thermoplastic-elastomers).

The skilled artisan will appreciate that the container means set forth above are by no means an exhaustive list, but merely serve as guidance to the artisan with respect to the variety of container means which are used to contain, hold, mix, blend, dispense, inject, transfer, nebulize, etc. an immunogen or immunogenic composition during research, processing, development, formulation, manufacture, storage and/or administration of the composition. Additional container means contemplated for use in the present invention may be found in published catalogues from laboratory equipment vendors and manufacturers such as United States Plastic Corp. (Lima, Ohio), VWR (West Chester, Pa.), BD Biosciences (Franklin Lakes, N.J.), Fisher Scientific International Inc. (Hampton, N.H.) and Sigma-Aldrich (St. Louis, Mo.).

Thus, the novel formulations of the present invention are particularly advantageous in that they stabilize and inhibit precipitation of immunogenic formulations comprised in a container means throughout the various stages of research, processing, development, formulation, manufacture, storage and/or administration of the composition. The novel formulations of the invention not only stabilize immunogenic compositions against physical/thermal stresses (e.g., temperature, humidity, shear forces, etc.), they also enhance stability and inhibit precipitation of immunogenic compositions against negative factors or influences such as incompatibility of the immunogenic composition with the container/closure system (e.g., a siliconized container means).

The stability of an immunogenic composition of the invention is readily determined using standard techniques, which are well known and routine to those of skill in the art. For example, an immunogenic composition is assayed for stability, aggregation, immunogenicity, particulate formation, protein (concentration) loss, and the like, by methods including, but not limited to, light scattering, optical density, sedimentation velocity centrifugation, sedimentation equilibrium centrifugation, circular dichroism (CD), Lowry assay, bicinchoninic acid (BCA) assay, antibody binding, and the like.

Having described various embodiments of the invention with reference to the accompanying description and drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLES

### Example 1

### Preparation of S. pneumoniae Capsular Polysaccharides

Methods of culturing pneumococci are well known in the art. See, e.g., Chase, 1967, Methods of Immunology and Immunochemistry 1:52. Methods of preparing pneumococcal capsular polysaccharides are also well known in the art. See, e.g., European Patent No. EP0497524. Isolates of pneumococcal subtypes are available from the ATCC.

The bacteria are identified as encapsulated, non-motile, Gram-positive, lancet-shaped diplococci that are alpha-hemolytic on blood-agar. Subtypes are differentiated on the basis of Quelling reaction using specific antisera. See, e.g., U.S. Pat. No. 5,847,112.

### Cell Banks

Cell banks representing each of the S. pneumococcus serotypes present in PCV-15 were obtained from the Merck Culture Collection (Rahway, N.J.) in a frozen vial.

### Inoculation

A thawed seed culture was transferred to the seed fermentor containing an appropriate pre-sterilized growth media.

### Seed Fermentation

The culture was grown in the seed fermentor with temperature and pH control. The entire volume of the seed fermentor was transferred to the production fermentor containing pre-sterilized growth media.

### Production Fermentation

The production fermentation was the final cell growth stage of the process. Temperature, pH and the agitation rate was controlled.

### Inactivation

The fermentation process was terminated via the addition of an inactivating agent. After inactivation, the batch was transferred to the inactivation tank where it was held at controlled temperature and agitation.

### Purification

Cell debris was removed using a combination of centrifugation and filtration. The batch was ultrafiltered and diafiltered. The batch was then subjected to solvent-based fractionations that remove impurities and recover polysaccharide.

### Example 2

### Preparation of Pneumococcal Polysaccharide-CRM₁₉₇ Conjugates

### Activation Process

The different serotype saccharides are individually conjugated to the purified CRM₁₉₇ carrier protein using a common process flow. In this process the saccharide is dissolved, sized to a target molecular mass, chemically activated and buffer-exchanged by ultrafiltration. The purified CRM₁₉₇ is then conjugated with the activated saccharide and the resulting conjugate is purified by ultrafiltration prior to a final 0.2 μm membrane filtration. Several process parameters within each step, such as pH, temperature, concentration, and time are serotype-specific as described in this example.

Step 1: Dissolution

Purified polysaccharide was dissolved in water to a concentration of 2-3 mg/mL. The dissolved polysaccharide was passed through a mechanical homogenizer with pressure preset from 0-1000 bar. Following size reduction, the saccharide was concentrated and diafiltered with sterile water on a 10 kDa MWCO ultrafilter. The permeate was discarded and the retentate was adjusted to a pH of 4.1 with a sodium acetate buffer, 50 mM final concentration. For serotypes 4 and 5, 100 mM sodium acetate at pH 5.0 was used. For serotype 4, the solution was incubated at 50°±2° C. Hydrolysis was stopped by cooling to 20-24° C.

Step 2: Periodate Reaction

The required sodium periodate molar equivalents for pneumococcal saccharide activation was determined using total saccharide content. With thorough mixing, the oxidation was allowed to proceed between 3-20 hours at 20-24° C. for all serotypes except 5, 7F, and 19F for which the temperature was 2-6° C.

Step 3: Ultrafiltration

The oxidized saccharide was concentrated and diafiltered with 10 mM potassium phosphate, pH 6.4 (10 mM sodium acetate, pH 4.3 for serotype 5) on a 10 kDa MWCO ultrafilter. The permeate was discarded and the retentate was adjusted to a pH of 6.3-8.4 by addition of 3 M potassium phosphate buffer.

### Conjugation Process

Step 1: Conjugation Reaction

The concentrated saccharide was mixed with CRM₁₉₇ carrier protein in a 0.2-2 to 1 charge ratio. The blended saccharide-CRM₁₉₇ mixture was filtered through a 0.2 μm filter.

The conjugation reaction was initiated by adding a sodium cyanoborohydride solution to achieve 1.8-2.0 moles of sodium cyanoborohydride per mole of saccharide. The reaction mixture was incubated for 48-120 hours at 20-24° C. (8-12° C. for serotypes 3, 5, 6A, 7F, 19A, and 19F).

Step 2: Borohydride Reaction

At the end of the conjugation incubation the reaction mixture was adjusted to 4-8° C., and a pH of 8-10 with either 1.2 M sodium bicarbonate buffer or 3 M potassium phosphate buffer (except serotype 5). The conjugation reaction was stopped by adding the sodium borohydride solution to achieve 0.6-1.0 moles of sodium borohydride per mole of saccharide (0 moles of borohydride added for serotype 5). The reaction mixture was incubated for 45-60 minutes.

Step 3: Ultrafiltration Steps

The reaction mixture was diafiltered on a 100 kDa MWCO ultrafilter with a minimum of 20 volumes of 100 mM potassium phosphate, pH 8.4 buffer. The retentate from the 100 kDa ultrafilter was diafiltered on a 300 kDa MWCO ultrafilter with a minimum of 20 diavolumes of 150 mM sodium chloride at 20-24° C. The permeate was discarded.

Step 4: Sterile Filtration

The retentate from the 300 kDa MWCO diafiltration was filtered through a 0.2 μm filter and filled into borosilicate glass containers at appropriate volumes for release testing, in-process controls, and formulation (except serotype 19F). The serotype 19F conjugate was passed through a 0.2 μm filter into a holding tank and incubated at 20-24° C. Following incubation, the conjugate was diafiltered on a 300 kDa MWCO ultrafilter with a minimum of 20 diavolumes of 150 mM sodium chloride at 20-24° C. The permeate was discarded, and the retentate was filtered through a 0.2 μm filter and filled into borosilicate glass containers at appropriate volumes for release testing, in-process controls, and formulation. The final bulk concentrates were stored at 2-8° C.

### Example 3

### Formulation of a 15-Valent Pneumococcal Conjugate Vaccine

The required volumes of bulk concentrates were calculated based on the batch volume and the bulk saccharide concentrations. The combined 15 conjugates were further diluted to a target adsorption concentration by the addition of excipients (e.g., poloxamer) which include sodium chloride, L-histidine, pH 5.8, containing buffer. After sufficient mixing, the blend was sterile filtered through a 0.2 μm membrane. The sterile formulated bulk was mixed gently during and following its blending with bulk aluminum phosphate. The formulated vaccine was stored at 2-8° C.

### Example 4

### Effect of Excipients on Agitation-Induced Aggregation

The stability of the 15-valent Pneumococcal Conjugate Vaccine-15 (PCV-15) was studied with various excipient and pH conditions after rotational agitation studies. PCV-15 was prepared in 20 mM histidine pH 5.8 and 150 mM sodium chloride with 0.25 mg/mL Aluminum Phosphate Adjuvant (APA). The CRM₁₉₇ protein at 64 μg/mL conjugated to Pneumococcal polysaccharide (PnP) Types 1, 3, 4, 5, 6A, 7F, 9V, 14, 18C, 19A, 19F, 22F, 23F, and 33F at 4 μg/mL and Type 68 at 8 μg/mL, for a total polysaccharide concentration of 64 μg/mL.

For the rotational agitation studies, the PCV-15 formulation backbone was prepared with the addition of surfactants and osmolytes, and surfactants in combination with osmolytes, at pH 5.8, 6.2, and 7.0. The sodium chloride concentration was adjusted from 150 mM to either 100 mM or 50 mM dependent upon the concentration of the osmolyte. The agitation studies were designed using rotational upright and side agitation for 24 hours at 4° C. Aggregation was observed visually and with Static Light Scattering.

Formulation Material:

PCV-15 formulation material was prepared as described in Examples 1-3. The formulated material was stored at 2-8° C. until all agitation studies were completed. The following formulations were prepared in 20 mM histidine with 0.25 mg/mL Aluminum Phosphate Adjuvant (APA) and 64 μg/mL polysaccharide:CRM conjugates:

1. 150 mM NaCl, pH 5.8

2. 150 mM NaCl, pH 6.0

3. 150 mM NaCl, pH 6.2

4. 150 mM NaCl, pH 6.6

5. 150 mM NaCl, pH 6.8

6. 150 mM NaCl, pH 7.0

7. 3% sucrose, 100 mM NaCl, pH 5.8

8. 3% sucrose, 100 mM NaCl, 0.02% PS-80, pH 5.8

9. 6% sucrose, 50 mM NaCl, pH 5.8

10. 6% sucrose, 50 mM NaCl, 0.02% PS80, pH 5.8

11. 3% sucrose, 100 mM NaCl, pH 6.2

12. 3% sucrose, 100 mM NaCl, 0.02% PS-80, pH 6.2

13. 6% sucrose, 50 mM NaCl, pH 6.2

14. 6% sucrose, 50 mM NaCl, 0.02% P580, pH 6.2

15. 6% trehalose, 50 mM NaCl, 0.02% PS-80, pH 6.2

16. 6% sucrose, 50 mM NaCl, 0.1% poloxamer 188, pH 6.2

17. 0.5% sucrose, 150 mM NaCl, 0.02% PS-80, pH 5.8

18. 0.5% sucrose, 150 mM NaCl, 0.1% poloxamer 188, pH 5.8

19. 0.5% sucrose, 150 mM NaCl, 0.02% PS-80, pH 5.8

20. 0.5% trehalose, 150 mM NaCl, 0.02% PS-80, pH 5.8

21. 0.5% sucrose, 150 mM NaCl, 0.02% PS-20, pH 5.8

22. 0.5% trehalose, 150 mM NaCl, 0.02% PS-20, pH 5.8

23. 0.5% sucrose, 150 mM NaCl, pH 5.8

24. 0.5% trehalose, 150 mM NaCl, pH 5.8

25. 150 mM NaCl, 0.02% PS-80, pH 5.8

26. 150 mM NaCl, 0.02% PS-20, pH 5.8

27. 150 mM NaCl, 0.02% PS-80, pH 6.2

28. 150 mM NaCl, 0.1% poloxamer 188, pH 5.8

29. 150 mM NaCl, 0.1% poloxamer 188, pH 6.2

30. 150 mM NaCl, 0.04% CTAB, pH 5.8

31. 50 mM NaCl, 6% sucrose, 0.1% poloxamer 188, pH 7.0

32. 150 mM NaCl, 0.1% poloxamer 188, pH 7.0

Agitation Study Procedures:

The purpose of the agitation study is to subject the vials to agitation conditions and then determine the effect those conditions have on aggregate formation. The study represents a direct agitation of the PCV-15 formulation through interactions with a hydrophilic surface (non-grinding) and exposure of the formulation to final container components and an air interface. The PCV-15 formulation was filled in non-sulfate-treated 2 mL vials at a fill volume of 0.75 mL with a 13 mm stopper.

For the rotational, vibrational, and end-over-end agitation methods, vials were agitated upright and side at 4° C. and 25° C. for 24 hours. The vials were attached directly to the agitation instruments except for upright rotational agitation in which the vials were placed in a 7×7 freezer box first. A lab-scale multi-purpose rotator at the maximum speed was used for rotational agitation while a digital vortex at 1,500 rpm was used for vibrational agitation. A rota-shake genie at the maximum speed was used for the end-over-end agitation. Observations were made at one hour intervals up to eight hours for the rotational agitation method and up to nine hours for the vibrational and end-over-end agitation methods with a final observation recorded at 24 hours for all three agitation methods. Vials were agitated in duplicate for the rotational method and in singlet for the vibrational and end-over-end methods due to limited availability of formulation material. All vials were compared to a non-agitated vial as the control.

Additionally for the rotational agitation method, 3% sulfate-treated 2 mL vials were filled with 0.75 mL of formulation. The vials were agitated at maximum speed for 24 hours upright and side and packaged with and without a 10-bi product carton at 4° C. for comparison to non-sulfate-treated vials agitated under the same conditions. A 10-bi product carton is one type of final marketed product package which can hold up to 10 vials and contains one product circular.

Results

After agitating the PCV-15 formulation 1 at pH 5.8, 6.0, 6.2, 6.6, 6.8, and 7.0 it was determined that a change in pH was not significant enough to prevent the formation of aggregates, specifically, after side agitation. The addition of the surfactant PS-80 individually and in combination with the osmolyte sucrose did prevent upright and side agitation-induced aggregation in some agitation studies, but not in all agitation studies. The addition of the surfactant PS-20 individually to the V114 formulation also prevented upright and side agitation-induced aggregation in some agitation studies, but not in all agitation studies. However, when PS-20 was added with the osmolyte sucrose or trehalose at pH 5.8, upright and side agitation-induced aggregation was prevented. Finally, the surfactant poloxamer 188 did prevent agitation induced aggregation individually and in combination with the osmolyte sucrose at pH 5.8, 6.2, and 7.0.

### CONCLUSIONS

After completion of the agitation studies using various surfactant, osmolyte, and pH conditions, the surfactant PS-20 was able to prevent agitation-induced aggregation in combination with the osmolytes sucrose and trehalose at pH 5.8. However, the surfactant poloxamer 188 was able to prevent agitation-induced aggregation individually and in combination with the osmolyte sucrose regardless of pH.

### Example 5

### Effect of Temperature, pH, Salt Concentration and Sucrose on Agitation-Induced Aggregation

The impact of rotational agitation insult on PCV-15 with several formulations following thermal stress was studied. For the rotational agitation studies, PCV-15 was prepared in several formulations by varying pH, salt concentration and adding sucrose (Table 1). Then these formulations were divided into three groups: 4° C.; 37° C. stress for 1 week, and 25° C. stress for 45 days. The 4° C. group were subjected to agitation immediately, while the other two groups were incubated according to the above conditions, then subjected to agitation. The agitation studies were designed using rotational side agitation for 24 hours at 4° C. Visual observations were used for all samples to detect particulates and the 4° C. and 37° C. samples were tested with Static Light Scattering to look at the particle size distribution in the samples.

TABLE 1
Formulations prepared for Agitation Study

20 mM Histidine pH 5.8, 150 mM NaCl, and 250 μg/ml APA
20 mM Histidine pH 5.8, 0.1% (w/v) Poloxamer 188, 150 mM NaCl,
and 250 μg/ml APA
20 mM Histidine pH 7.0, 0.1% (w/v) Poloxamer 188, 150 mM NaCl,
and 250 μg/ml APA
20 mM Histidine pH 7.0, 0.1% (w/v) Poloxamer 188, 6% (w/v)
Sucrose, 50 mM NaCl, and 250 μg/ml APA

Methods:

Preparation of Formulations:

Formulations were prepared aseptically in a class II Biosafety cabinet from polysaccharide-protein conjugates as described above that were stored in 10 mM Histidine pH 7.0 and saline and then formulated as defined in Table 1.

Rotational Agitation:

This study represents a direct agitation of the PCV-15 formulation through interactions with a hydrophilic surface (non-grinding) and exposure of the formulation to final container components and an air/liquid interface. The PCV-15 formulation was filled in non-sulfate-treated 2 mL vials at a fill volume of 0.75 mL with a 13 mm stopper. The vials were subjected to thermal stress conditions, if necessary, then agitated on the side at 4° C. for 24 hours. A lab-scale multi-purpose rotator at the maximum speed was used for rotational agitation. The vials were attached directly to the rotator for side agitation. Observations were made at 24 hours.

Results:

PCV-15 formulations (Table 1) following rotational agitation in vials for 24 hrs at 4° C. resulted in formation of precipitates in the absence of Poloxamer 188, regardless of stress conditions. The addition of Poloxamer 188 resulted in no observed particulates (Table 2). 15 vials were examined for each condition.

A control experiment was performed with 20 mM Histidine pH 5.8, 150 mM NaCl at 4° C., in the presence or absence of APA. Results were comparable showing that the presence of APA did not contribute to the inhibition of agitation-induced aggregation.

TABLE 2
Impact of Surfactants on Agitation Induced
Aggregation formation in Vials

| | Appearance |
|---|---|
| Formulation | (Visual Inspection) |
| 20 mM Histidine pH 5.8 and 150 mM NaCl | Particulates |
| 4° C. | |
| 20 mM Histidine pH 5.8 and 150 mM NaCl | Particulates |
| 25° C. 45 Days | |
| 20 mM Histidine pH 5.8 and 150 mM NaCl | Particulates |
| 37° C. 1 week | |
| 20 mM Histidine pH 5.8, 0.1% (w/v) Poloxamer | None |
| 188 and 150 mM NaCl 4° C. | |
| 20 mM Histidine pH 5.8, 0.1% (w/v) Poloxamer | None |
| 188 and 150 mM NaCl 25° C. 45 Days | |
| 20 mM Histidine pH 5.8, 0.1% (w/v) Poloxamer | None |
| 188 and 150 mM NaCl 37° C. 1 week | |
| 20 mM Histidine pH 7.0, 0.1% (w/v) Poloxamer | None |
| 188 and 150 mM NaCl 4° C. | |
| 20 mM Histidine pH 7.0, 0.1% (w/v) Poloxamer | None |
| 188 and 150 mM NaCl 25° C. 45 Days | |
| 20 mM Histidine pH 7.0, 0.1% (w/v) Poloxamer | None |
| 188 and 150 mM NaCl 37° C. 1 week | |
| 20 mM Histidine pH 7.0, 0.1% (w/v) Poloxamer | None |
| 188, 6% (w/v) Sucrose and 50 mM NaCl 4° C. | |
| 20 mM Histidine pH 7.0, 0.1% (w/v) Poloxamer | None |
| 188, 6% (w/v) Sucrose and 50 mM NaCl 25° C. | |
| 45 Days | |
| 20 mM Histidine pH 7.0, 0.1% (w/v) Poloxamer | None |
| 188, 6% (w/v) Sucrose and 50 mM NaCl 37° C. | |
| 1 week | |

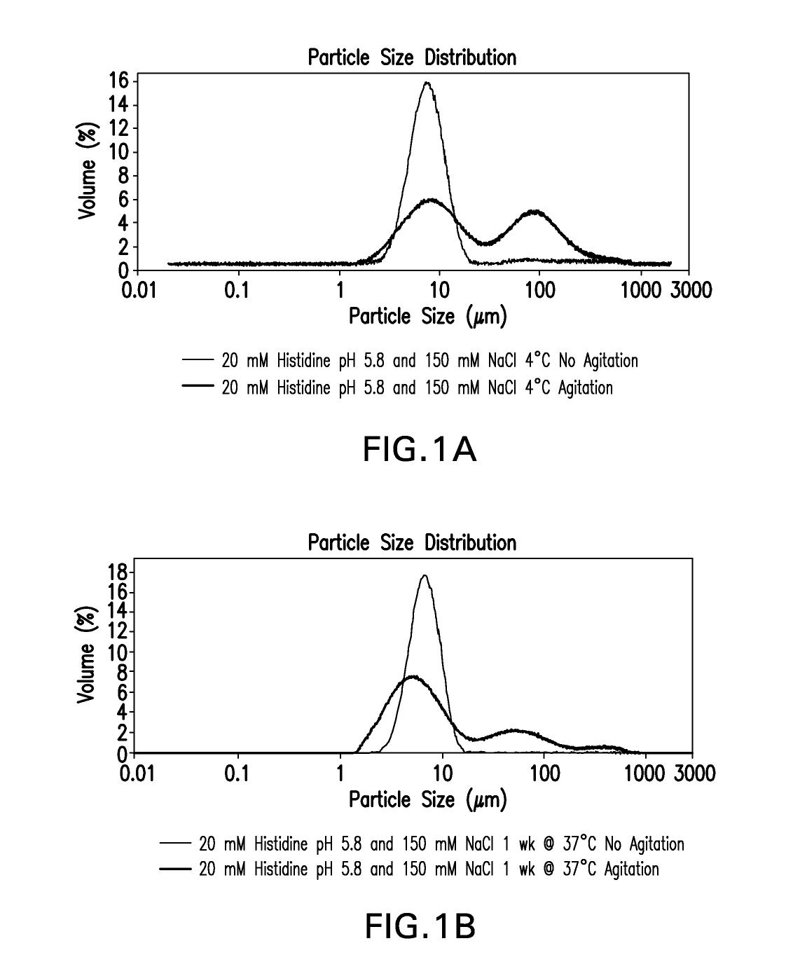

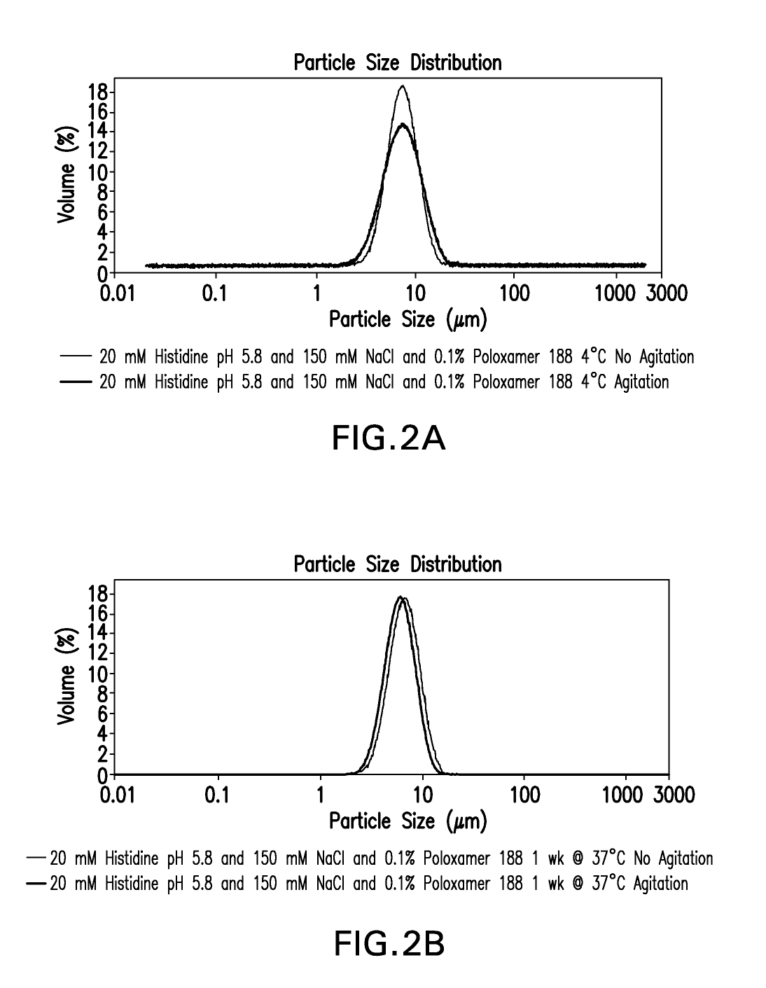

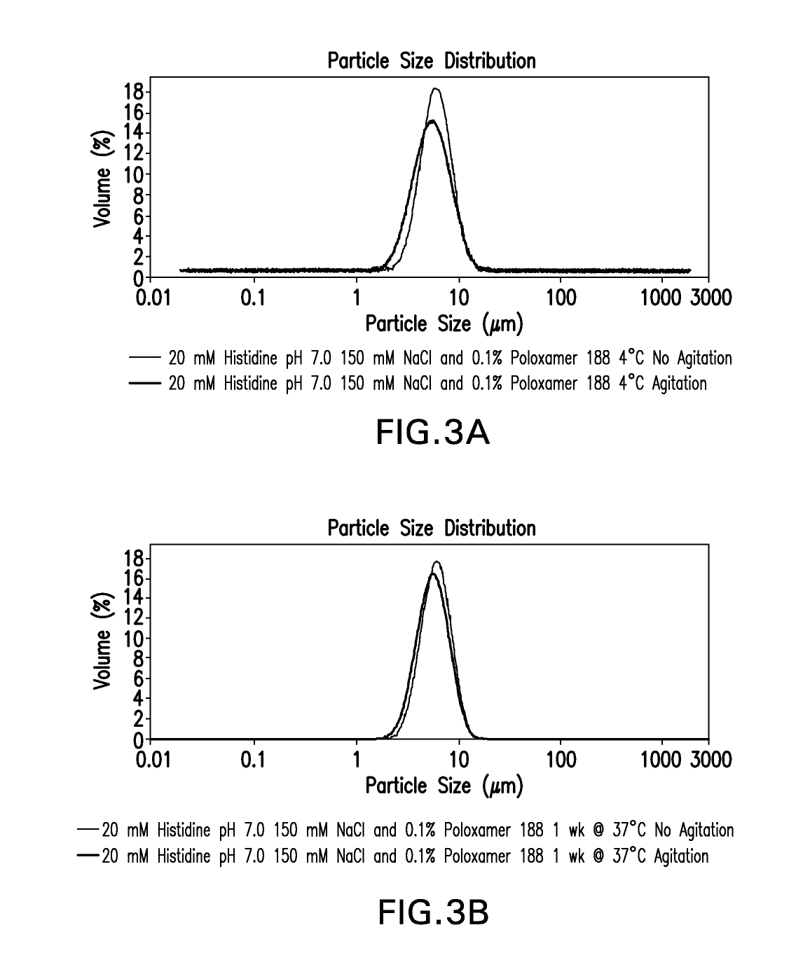

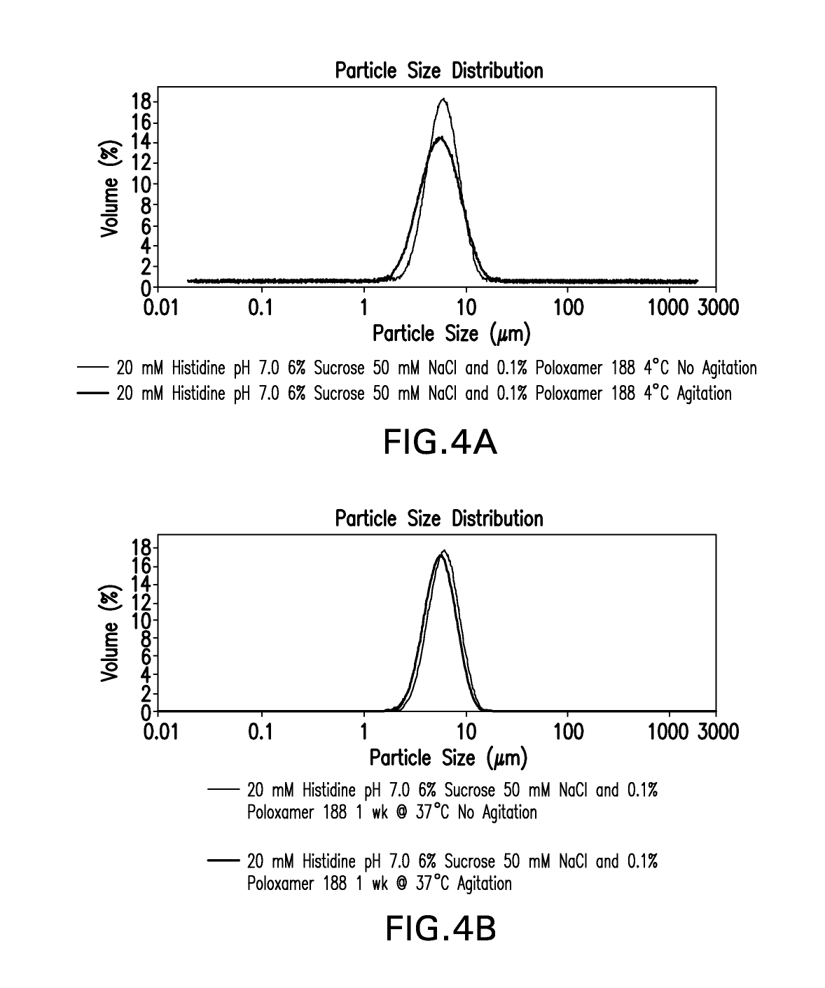

Static light scattering was also performed on the 4° C. and 37° C. samples to look at size distributions. In formulations without poloxamer 188 a population of larger particulates was observed. Formulations containing poloxamer 188 only had one population of particles that were comparable to the populations seen in the non-agitated samples (FIGS. 1A-4B).

### CONCLUSIONS

Poloxamer 188 at a final concentration of 0.1% was able to mitigate agitation-induced aggregation in vials, even upon thermally stressing the formulations prior to agitation.

### Example 6

### Impact of Surfactants on Prevention of Agitation-Induced Aggregation During ISTA Standard Testing

### Introduction

The purpose of the ISTA standard 3A study is to expose the vial formulation to the vibrational stress and potential drop stress observed during routine shipping. For the studies, PCV15 was prepared in several formulations then subjected to the ISTA 3A procedure. See International Safe Transit Association Procedure 3A (East Lansing, Mich.). Utilizing the ISTA standard process, results would be more in-line with the expected conditions material would experience when shipped to the developed world. Samples were then stored at 2-8° C. Following storage at 2-8° C., samples were visually inspected for detection of particulates and then additionally analyzed by static light scattering (SLS) for particle size distribution.

Methods:

Preparation of Formulations:

Formulations were prepared aseptically in a class II Biosafety cabinet from polysaccharide-protein conjugates as described above that were stored in 10 mM Histidine pH 7.0 and saline and then formulated as defined in Table 3.

TABLE 3
Formulations prepared for Agitation Study

20 mM Histidine pH 5.8, 150 mM NaCl, and 250 μg/ml APA
20 mM Histidine pH 5.8, 150 mM NaCl, 0.02% PS80 and 250
μg/mL APA
20 mM Histidine pH 5.8, 150 mM NaCl, 0.02% PS20 and 250
μg/mL APA
20 mM Histidine pH 5.8, 150 mM NaCl, 0.1% Poloxamer 188
and 250 μg/mL APA
20 mM Histidine pH 6.2, 6% Sucrose, 50 mM NaCl, 0.02% PS80
and 250 μg/mL APA
20 mM Histidine pH 6.2, 3% Sucrose, 100 mM NaCl, 0.02% PS80
and 250 μg/mL APA
20 mM Histidine pH 6.2, 150 mM NaCl, 0.02% PS80 and 250
μg/mL APA
20 mM Histidine pH 6.2, 6% Trehalose, 50 mM NaCl, 0.02%
PS80 and 250 μg/mL APA
20 mM Histidine pH 6.2, 150 mM NaCl, 0.1% Poloxamer 188
and 250 μg/mL APA
20 mM Histidine pH 6.2, 6% Sucrose, 50 mM NaCl, 0.1%
Poloxamer 188 and 250 μg/mL APA
20 mM Histidine pH 5.8, 0.5% Sucrose, 150 mM NaCl, 0.02%
PS80
20 mM Histidine pH 5.8, 0.5% Sucrose, 150 mM NaCl, 0.1%
Poloxamer 188

ISTA Standard Agitation Study:

The vials were subjected to the ISTA 3A procedure.

Materials used were those specified in the ISTA procedure 3A and include gel shipper base (base to an Expanded Polystyrene (EPS) thermal shipper); PolarPack gel, 28 ounces (Refrigerant used to maintain product temperature); PCS 50913 TempTale monitor with a 0° C. low temperature alarm and a 25° C. high temperature alarm; PCS 50900 Corrugated pad, slotted; Tape Scotch tape and packing tape used to hold product cartons and gel shipper shut, respectively; 10× product carton (Carton designed to hold 10 stoppered and crimped vials in a 5×2 pattern with a product circular separating the 2 rows of 5); and PCS 50837 Gel shipper lid (Lid made of EPS).

The following carton preparation was performed:

Assembled 1 10× product carton by taping I side shut with scotch tape.

Defaced the 10× product carton on all sides with a black permanent marker.

Obtained 10 vials of PCV 15 formulations.

Placed all 10 vials of V114 DP formulations into the ProQuad 10× product carton.

Taped the open end of the 10× product carton shut.
Placed the packaged 10× product carton inside the gel shipper between 4 refrigerated PolarPak gels, 28 ounce, 2 gels on bottom and 2 gels on top.

Immediately after carton preparation was completed the material was transported to Tegrant Corporation (Montgomeryville, Pa.) for storage/pre test conditioning. All materials were placed into the following temperature conditions until testing was conducted: PolarPack gel refrigerant, refrigerated 5° C.±3° C., Vials of PCV 15 formulations 5° C.±3° C.; PolarPack gel refrigerant, frozen −20° C.±5° C., All other components at room temperature. All materials were in these temperature storage conditions for over 24 hours.

The following pack out was performed at Tegrant Corporation:

Obtained gel shipper base.
Placed 1 refrigerated PolarPack gel, 28 ounces flat in the bottom of the gel shipper base.
Placed 1 TempTale on top of the PolarPack gel touching an edge of the gel shipper inner wall, turned it on.
Placed 1 corrugated pad, slotted on top of the refrigerated PolarPack gel and TempTale.

Placed the PCV 15 formulation filled product cartons on top of the corrugated pad.

Placed 1 TempTale in the top layer of PCV 15 formulation filled product cartons.
Placed 1 corrugated pad, slotted on top of the PCV 15 formulation filled product cartons.
Placed 1 TempTale in the of the corrugated pad touching the opposite edge of the gel shipper wall as the bottom, turn it on.
Placed 1 refrigerated PolarPack gel, 28 ounces flat on top of the corrugated pad and TempTale.
Placed 2 frozen PolarPack gels, 28 ounces, flat on top of the refridgerated PolarPack gel.

Placed gel shipper lid on gel shipper base.

Taped the gel shipper shut with packing tape.

The packed out gel shipper was transported to Mid-Atlantic Packaging (Montgomeryville, Pa.) for distribution testing according to the ISTA 3A test procedure.

In addition to the ISTA testing, a subset of vials was also exposed to rotational shake conditions previously described. A lab-scale multi-purpose rotator at the maximum speed was used for rotational agitation. The vials were attached directly to the rotator for side agitation. Observations were made at 24 hours.

Results:

Rotational Agitation Results:

PCV15 formulations following rotational agitation in vials for 24 hrs at 4° C. resulted in formation of precipitates in the absence of Poloxamer 188. The addition of the surfactant, Poloxamer 188, resulted in no observed particulates (Table 4).

TABLE 4
Impact of Surfactants on Agitation Induced Aggregation formation in Vials

| | Appearance |
|---|---|
| Formulation | (Visual Inspection) |
| 20 mM Histidine pH 5.8, 150 mM NaCl, and 250 | Moderate Amount of small stringy |
| μg/ml APA | aggregates |
| 20 mM Histidine pH 5.8, 150 mM NaCl, 0.02% | Small aggregation on side few small |
| PS80 and 250 μg/mL APA | aggregates |
| 20 mM Histidine pH 5.8, 150 mM NaCl, 0.02% | Very small aggregation on side two very |
| PS20 and 250 μg/mL APA | small aggregates |
| 20 mM Histidine pH 5.8, 150 mM NaCl, 0.1% | No aggregates |
| Poloxamer 188 and 250 μg/mL APA | |
| 20 mM Histidine pH 6.2, 6% Sucrose, 50 mM NaCl, | Small aggregation on side |
| 0.02% PS80 and 250 μg/mL APA | |
| 20 mM Histidine pH 6.2, 3% Sucrose, 100 mM | Small aggregation on side few small to |
| NaCl, 0.02% PS80 and 250 μg/mL APA | moderate aggregates |
| 20 mM Histidine pH 6.2, 150 mM NaCl, 0.02% | Small aggregation on side very few small |
| PS80 and 250 μg/mL APA | aggregates |
| 20 mM Histidine pH 6.2, 6% Trehalose, 50 mM | Small aggregation on side very few small |
| NaCl, 0.02% PS80 and 250 μg/mL APA | aggregates |
| 20 mM Histidine pH 6.2, 150 mM NaCl, 0.1% | No aggregates |
| Poloxamer 188 and 250 μg/mL APA | |
| 20 mM Histidine pH 6.2, 6% Sucrose, 50 mM NaCl, | No aggregates |
| 0.1% Poloxamer 188 and 250 μg/mL APA | |
| 20 mM Histidine pH 5.8, 0.5% Sucrose, 150 mM | Very small aggregates on side very few |
| NaCl, 0.02% PS80 | small aggregates |
| 20 mM Histidine pH 5.8, 0.5% Sucrose, 150 mM | No aggregates |
| NaCl, 0.1% Poloxamer 188 | |

ISTA Standard Testing Results:

V114 formulations (Table 3) following the ISTA standard 3A procedure were examined visually for aggregates. Formulations containing Poloxamer 188 or Polysorbate (PS) mitigated the appearance of aggregates (Table 5).

TABLE 5
Impact of Surfactants on Agitation Induced Aggregation formation
in Vials Following Exposure to the ISTA 3A Procedure

| | Appearance |
|---|---|
| Formulation | (Visual Inspection) |
| 20 mM Histidine pH 5.8, 150 mM NaCl, and 250 | Many small |
| μg/ml APA | aggregates |
| 20 mM Histidine pH 5.8, 150 mM NaCl, 0.02% | No Aggregates |
| PS80 and 250 μg/mL APA | |
| 20 mM Histidine pH 5.8, 150 mM NaCl, 0.02% | No Aggregates |
| PS20 and 250 μg/mL APA | |
| 20 mM Histidine pH 5.8, 150 mM NaCl, 0.1% | No Aggregates |
| Poloxamer 188 and 250 μg/mL APA | |
| 20 mM Histidine pH 6.2, 6% Sucrose, 50 mM | No Aggregates |
| NaCl, 0.02% PS80 and 250 μg/mL APA | |
| 20 mM Histidine pH 6.2, 3% Sucrose, 100 mM | No Aggregates |
| NaCl, 0.02% PS80 and 250 μg/mL APA | |
| 20 mM Histidine pH 6.2, 150 mM NaCl, 0.02% | No Aggregates |
| PS80 and 250 μg/mL APA | |
| 20 mM Histidine pH 6.2, 6% Trehalose, 50 mM | No Aggregates, |
| NaCl, 0.02% PS80 and 250 μg/mL APA | Difficult to Resuspend |
| 20 mM Histidine pH 6.2, 150 mM NaCl, 0.1% | No Aggregates |
| Poloxamer 188 and 250 μg/mL APA | |
| 20 mM Histidine pH 6.2, 6% Sucrose, 50 mM | No Aggregates |
| NaCl, 0.1% Poloxamer 188 and 250 μg/mL APA | |
| 20 mM Histidine pH 5.8, 0.5% Sucrose, 150 mM | No Aggregates |
| NaCl, 0.02% PS80 | |
| 20 mM Histidine pH 5.8, 0.5% Sucrose, 150 mM | No Aggregates |
| NaCl, 0.1% Poloxamer 188 | |

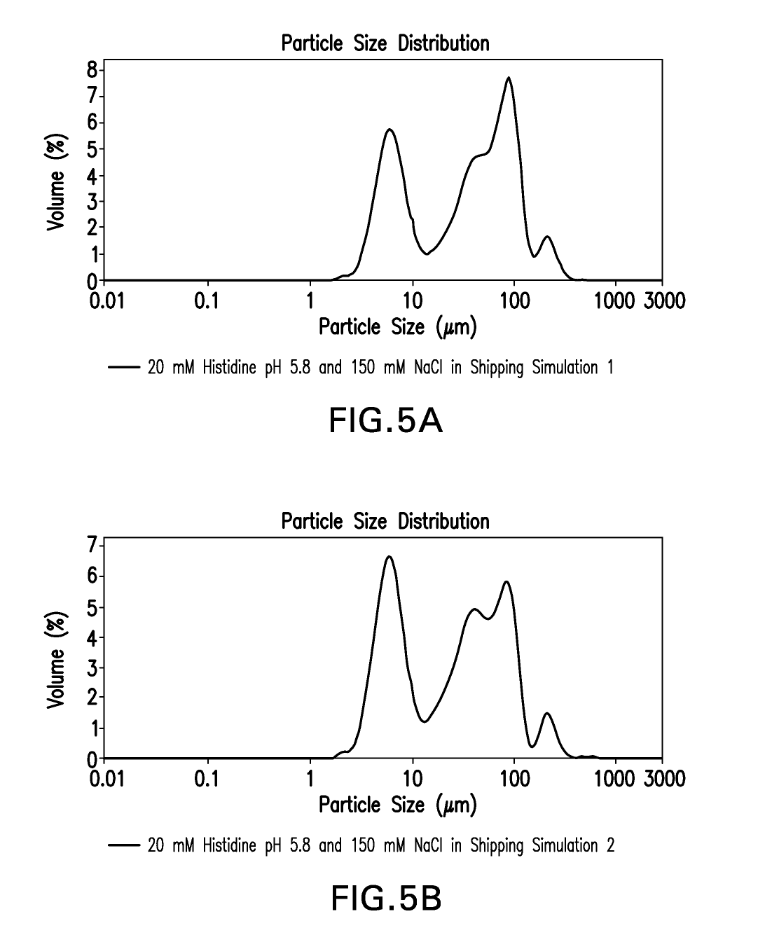

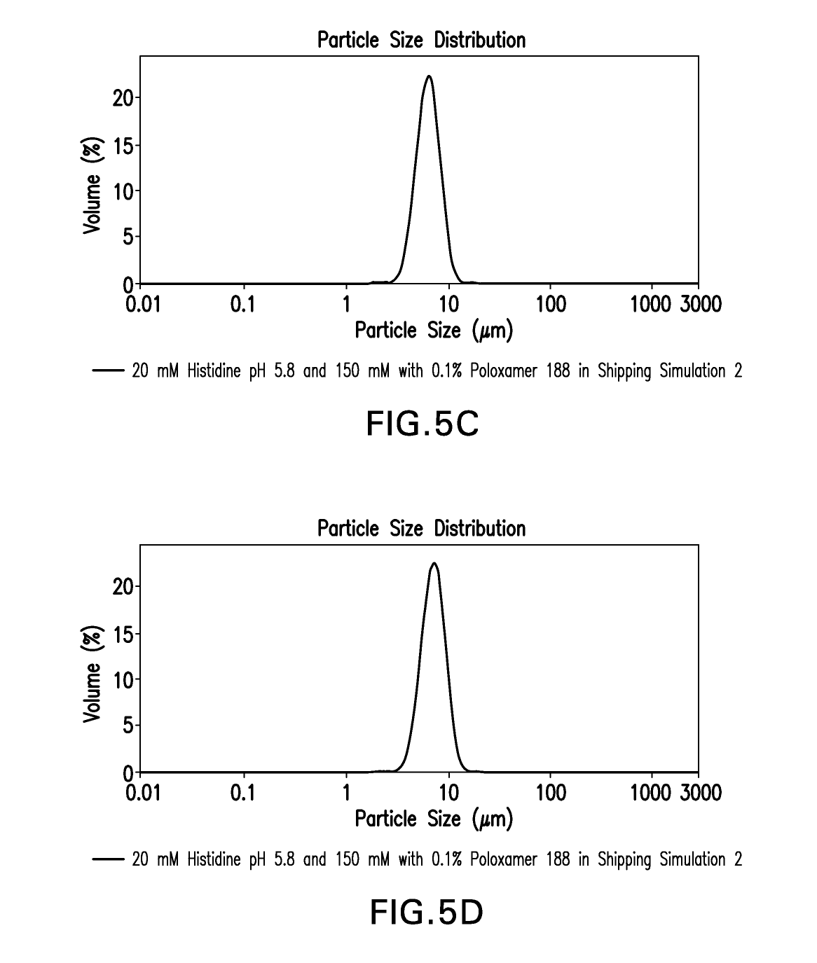

In addition to the visual inspection, static light scattering was also performed on a subset of the samples to examine size distributions. The control formulation without surfactant led to both increased particle size, as well as, a broader distribution of particles following the ISTA testing (FIGS. 5A-B). While formulations containing Poloxamer 188 showed a more homogenous product after exposure to the ISTA testing (FIGS. 5C-D).

### CONCLUSIONS

Multiple formulations were exposed to both rotational and vibrational stress and observed visually as well as utilizing static light scattering for particle size distribution. Results indicate that the addition of a surfactant (polysorbate or Poloxamer 188) can mitigate agitation-induced aggregation caused by vibrational stress (ISTA standard 3A results). However, only Poloxamer 188 can mitigate both vibrational and rotational stress associated with the V 114 formulation.

### Example 7

### Poloxamer Range Finding

PCV-15 was prepared as described in Examples 1 to 3 as a vial image with of 20 mM histidine pH 5.8 and 150 mM sodium chloride with 0.25 mg/mL Aluminum Phosphate Adjuvant (APA). For the rotational agitation studies, PCV-15 was prepared with the addition of surfactants on Aluminum Phosphate Adjuvant (APA). The agitation studies were designed using rotational side agitation for 24 hours at 4° C. A path of a beam of light (Tindel effect) passing through the vial allowed for the detection of particulates.

Methods:

Preparation of Formulations:

Formulations were prepared as described in Examples 1 to 3 aseptically in a class II Biosafety cabinet. Formulations were stored at 4° C. until initiation of agitation study.

TABLE 6
Formulations evaluated in this study

20 mM Histidine pH 5.8 and 150 mM NaCl
20 mM Histidine pH 5.8 and 150 mM NaCl + 0.025% poloxamer 188
20 mM Histidine pH 5.8 and 150 mM NaCl + 0.05% poloxamer 188
20 mM Histidine pH 5.8 and 150 mM NaCl + 0.1% poloxamer 188
20 mM Histidine pH 5.8 and 150 mM NaCl + 0.15% poloxamer 188
20 mM Histidine pH 5.8 and 150 mM NaCl + 0.02% PS80

Rotational Agitation:

The purpose of the agitation study is to subject the vials to agitation conditions and then determine the effect those conditions have on aggregate formation. The PCV-15 formulation was filled in non-sulfate-treated 2 mL vials at a fill volume of 0.75 mL with a 13 mm stopper. The vials were agitated side at 4° C. for 24 hours. A lab-scale multi-purpose rotator at the maximum speed was used for rotational agitation. The vials were attached directly to the rotator for side agitation.

Observations were made at 24 hours.

Results:

PCV15 Formulations after rotational agitation in vials for 24 hrs at 4° C. resulted in formation of precipitates in the absence of Poloxamer 188 or the presence of PS80. The addition of the surfactant Poloxamer 188 at a concentration between 0.025% and 0.15% resulted in no observed particulates. However, at the higher concentrations, 0.1% and 0.15%, the appearance of a slight haze (fanning) was observed on region of vial where air/liquid interface occurred (Table 7).

TABLE 7
Impact of Surfactants on Agitation Induced Aggregation
formation in Vials of PCV15 with Aluminum

| | | Appearance |
|---|---|---|
| Formulation | n | (Visual Inspection) |
| 20 mm Histidine pH 5.8 and 150 mM NaCl | 3 | Particulates |
| 20 mm Histidine pH 5.8 and 150 mM NaCl & | 3 | none |
| 0.025% poloxamer 188 | | |
| 20 mm Histidine pH 5.8 and 150 mM NaCl & | 3 | none |
| 0.05% poloxamer 188 | | |
| 20 mm Histidine pH 5.8 and 150 mM NaCl & | 3 | none/fanning |
| 0.1% poloxamer 188 | | |
| 20 mm Histidine pH 5.8 and 150 mM NaCl & | 3 | none/fanning |
| 0.15% poloxamer 188 | | |
| 20 mm Histidine pH 5.8 and 150 mM NaCl & | 3 | Particulates |
| 0.02% PS80 | | |

### CONCLUSIONS

The surfactant Poloxamer 188 was able to prevent agitation-induced aggregation in vials at a concentration between 0.025-0.15% (w/v).

### Example 8

### Effect of Preservatives on Agitation-Induced Aggregation

The stability of the 15-valent Pneumococcal Conjugate Vaccine-15 (PCV-15) was studied with various preservatives, alone or in combination with surfactant, after rotational agitation studies. PCV-15 was prepared in 20 mM histidine pH 5.8 and 150 mM sodium chloride with 0.25 mg/mL Aluminum Phosphate Adjuvant (APA). The CRM₁₉₇ protein at 64 μg/mL conjugated to Pneumococcal polysaccharide (PnP) Types 1, 3, 4, 5, 6A, 7F, 9V, 14, 18C, 19A, 19F, 22F, 23F, and 33F at 4 μg/mL and Type 6B at 8 μg/mL, for a total polysaccharide concentration of 64 μg/mL.

Rotational agitation studies were performed as described in Example 4. The PCV-15 formulation backbone was prepared with the addition of phenol, 2-phenoxyethanol, m-cresol, benzyl alcohol, or chlorobutanol at the specified concentrations, alone or in combination with Poloxamer 188. Some preservatives were dissolved in the specified solvent. The agitation studies were designed using rotational side agitation for 24 hours at 4° C. Aggregation was observed visually and, in some cases, with Static Light Scattering.

Results:

PCV 15 Formulations after rotational agitation in vials for 24 hrs at 4° C. generally resulted in formation of precipitates in the absence of Poloxamer 188. The addition of the surfactant 0.1% Poloxamer 188 resulted in no observed particulates with any of the preservatives tested.

TABLE 8
Impact of Preservatives on Agitation Induced Aggregation
formation in Vials of PCV15 with Aluminum

| Formulation | n | Temp. | Observations |
|---|---|---|---|
| 20 mM Histidine, 150 mM NaCl pH 5.8, 0.5% Phenol | 1 | 2-8 C. | Particulates |
| 20 mM Histidine, 150 mM NaCl pH 5.8, 1.0% Phenol | 1 | 2-8 C. | Particulates |
| 20 mM Histidine, 150 mM NaCl pH 5.8, 2.0% Phenol | 1 | 2-8 C. | Particulates |
| 20 mM Histidine, 150 mM NaCl, 0.3% Phenol, 0.1% | 1 | 2-8 C. | No Particulates |
| Poloxamer 188 pH 5.8 | | | |
| 20 mM Histidine, 150 mM NaCl, 0.6% Phenol, 0.1% | 1 | 2-8 C. | No Particulates |
| Poloxamer 188 pH 5.8 | | | |
| 20 mM Histidine, 150 mM NaCl, 0.5% Phenol, 0.1% | 2 | 2-8 C. | No Particulates |
| Poloxamer 188 pH 5.8 | 1 | 37 C. | No Particulates |
| 20 mM Histidine, 150 mM NaCl, 1.0% Phenol, 0.1% | 1 | 2-8 C. | Cloudy appearance, |
| Poloxamer 188 pH 5.8 | | | no particulates |
| 20 mM Histidine, 150 mM NaCl pH 5.8, 0.5% 2- | 1 | 2-8 C. | Particulates |
| Phenoxyethanol (1.4% Ethanol) | | | |
| 20 mM Histidine, 150 mM NaCl pH 5.8, 1.0% 2- | 1 | 2-8 C. | No Particulates |
| Phenoxyethanol (2.8% Ethanol) | | | |
| 20 mM Histidine, 150 mM NaCl pH 5.8, 1.3% 2- | 1 | 2-8 C. | No Particulates |
| Phenoxyethanol (3.6% Ethanol) | | | |
| 20 mM Histidine, 150 mM NaCl, 0.5% 2-Phenoxyethanol | 1 | 2-8 C. | No Particulates |
| (1.4% Ethanol), 0.1% Poloxamer 188 pH 5.8 | | | |
| 20 mM Histidine, 150 mM NaCl, 1.0% 2-Phenoxyethanol | 1 | 2-8 C. | No Particulates |
| (2.8% Ethanol), 0.1% Poloxamer 188 pH 5.8 | | | |
| 20 mM Histidine, 150 mM NaCl, 1.3% 2-Phenoxyethanol | 2 | 2-8 C. | No Particulates |
| (3.6% Ethanol), 0.1% Poloxamer 188 pH 5.8 | 1 | 37 C. | No Particulates |
| 20 mM Histidine, 150 mM NaCl pH 5.8, 0.5% m-cresol | 1 | 2-8 C. | Particulates |
| 20 mM Histidine, 150 mM NaCl, 0.1% m-cresol, 0.1% | 1 | 2-8 C. | No Particulates |
| Poloxamer 188 pH 5.8 | | | |
| 20 mM Histidine, 150 mM NaCl, 0.2% m-cresol, 0.1% | 1 | 2-8 C. | No Particulates |
| Poloxamer 188 pH 5.8 | | | |
| 20 mM Histidine, 150 mM NaCl, 0.3% m-cresol, 0.1% | 1 | 2-8 C. | No Particulates |
| Poloxamer 188 pH 5.8 | 1 | 37 C. | No Particulates |
| 20 mM Histidine, 150 mM NaCl 0.4% m-cresol, 0.1% | 1 | 2-8 C. | No Particulates |
| Poloxamer 188 pH 5.8 | | | |
| 20 mM Histidine, 150 mM NaCl, 0.5% m-cresol, 0.1% | 1 | 2-8 C. | Cloudy appearance, |
| Poloxamer 188 pH 5.8 | | | no particulates |
| 20 mM Histidine, 150 mM NaCl, 1.0% Benzyl Alcohol pH 5.8 | 1 | 2-8 C. | Particulates |
| 20 mM Histidine, 150 mM NaCl, 1.0% Benzyl Alcohol, | 2 | 2-8 C. | No Particulates |
| 0.1% Poloxamer 188 pH 5.8 | 1 | 37 C. | No Particulates |
| 20 mM Histidine, 150 mM NaCl, 0.5% Chlorobutanol (7.5% | 1 | 2-8 C. | Particulates |
| Ethanol) pH 5.8 | | | |
| 20 mM Histidine, 150 mM NaCl, 0.5% Chlorobutanol (7.5% | 2 | 2-8 C. | No Particulates |
| Ethanol), 0.1% Poloxamer 188 pH 5.8 | 1 | 37 C. | No Particulates |
| 20 mM Histidine, 150 mM NaCl, 0.5% CB (5% PEG300) pH 5.8 | 1 | 2-8 C. | Particulates |
| 20 mM Histidine, 150 mM NaCl, 0.1% P188, 0.5% CB | 1 | 2-8 C. | No Particulates |
| (5% PEG300) pH 5.8 | | | |

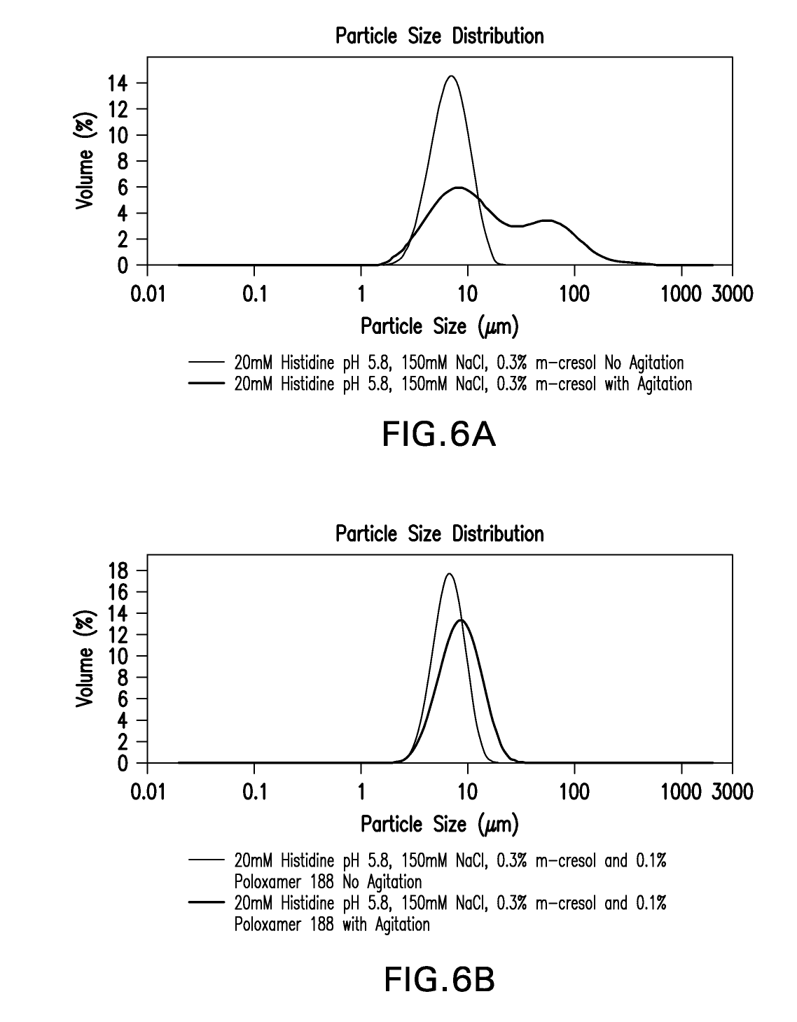

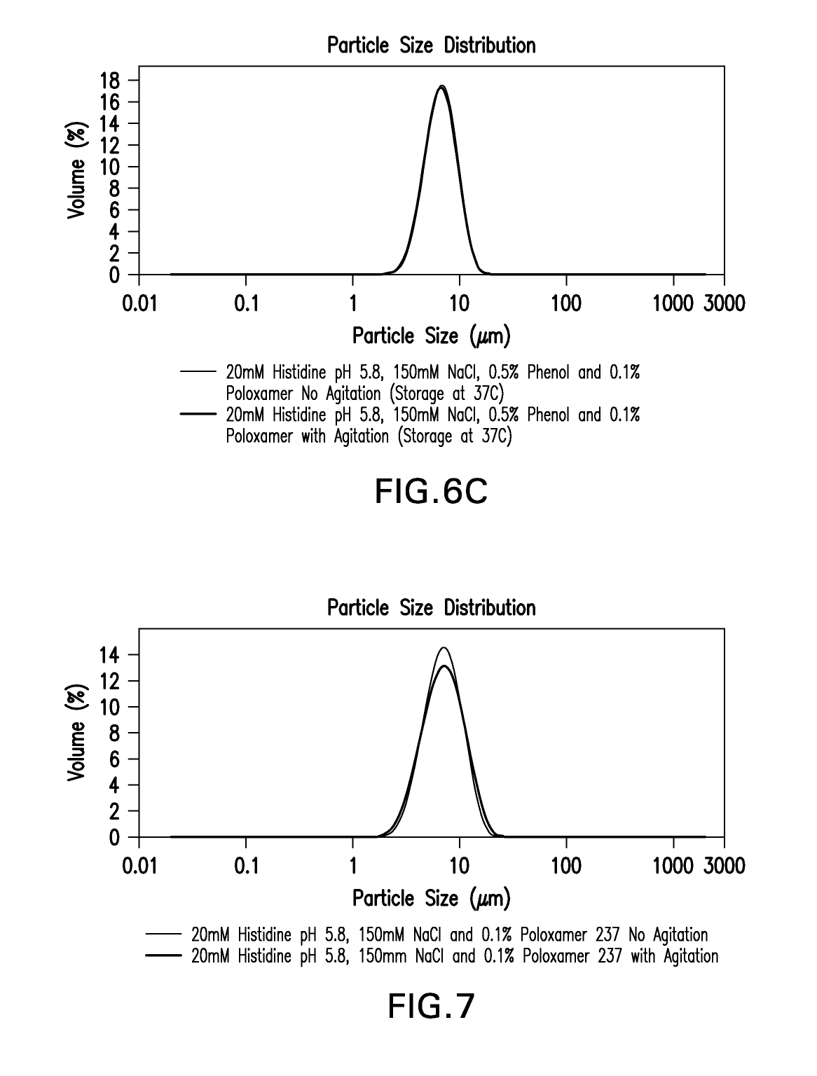

Static light scattering was also performed on representative samples to look at size distributions. In formulations without poloxamer 188 a population of larger particulates was observed. Formulations containing poloxamer 188 with either m-cresol or phenol had one population of particles that were comparable to the populations seen in the non-agitated samples (FIGS. 6A-C).

### CONCLUSIONS

The various preservatives tested had no detrimental effect on the ability of Poloxamer 188 to inhibit agitation-induced aggregation.

### Example 9

### Effect of pH and Buffers on Agitation-Induced Aggregation

The stability of the 15-valent Pneumococcal Conjugate Vaccine-15 (PCV-15) was studied with various buffer and pH conditions after rotational agitation studies. PCV-15 was prepared in 20 mM of the specified buffer and 150 mM sodium chloride with 0.25 mg/mL Aluminum Phosphate Adjuvant (APA). The CRM₁₉₇ protein at 64 μg/mL conjugated to Pneumococcal polysaccharide (PnP) Types 1, 3, 4, 5, 6A, 7F, 9V, 14, 18C, 19A, 19F, 22F, 23F, and 33F at 4 μg/mL and Type 6B at 8 μg/mL, for a total polysaccharide concentration of 64 μg/mL.

For the rotational agitation studies, the PCV-15 formulation backbone was prepared with the addition of surfactants and osmolytes, and surfactants in combination with osmolytes, at pH 5.2, 5.4, 5.6, 5.8, 6.0, 6.2, 6.6, 6.8, 7.0 and 8.0. The agitation studies were designed using rotational side agitation for 24 hours at 4° C. Aggregation was observed visually.

Results:

PCV15 Formulations after rotational agitation in vials for 24 hrs at 4° C. resulted in formation of precipitates in the absence of Poloxamer 188 for the buffer and pH conditions tested. The addition of the surfactant 0.1% Poloxamer 188 resulted in no observed particulates with any of the buffer and pH conditions tested.

TABLE 8
Impact of Buffer and pH Conditions on Agitation Induced
Aggregation formation in Vials of PCV15 with Aluminum

| Formulation | n | Temp. | Observations |
|---|---|---|---|
| 20 mM Histidine, 150 mM NaCl pH 5.2 | 1 | 2-8 C. | Particulates |
| 20 mM Histidine, 150 mM NaCl pH 5.4 | 1 | 2-8 C. | Particulates |
| 20 mM Histidine, 150 mM NaCl pH 5.6 | 1 | 2-8 C. | Particulates |
| 20 mM Histidine, 150 mM NaCl pH 5.8 | 1 | 2-8 C. | Particulates |
| 20 mM Succinate, 150 mM NaCl, 5.7 | 1 | 2-8 C. | Particulates |
| 20 mM Succinate, 150 mM NaCl, 6.0 | 1 | 2-8 C. | Particulates |
| 20 mM HEPES, 150 mM NaCl, pH 7.0 | 1 | 2-8 C. | Particulates |
| 20 mM Tris-HCl, 150 mM NaCl, pH 8.0 | 1 | 2-8 C. | Particulates |
| 20 mM Histidine, 150 mM NaCl pH 5.2 | 1 | 2-8 C. | No Particulates |
| with 0.1% Poloxamer 188 | | | |
| 20 mM Histidine, 150 mM NaCl pH 5.4 | 1 | 2-8 C. | No Particulates |
| with 0.1% Poloxamer 188 | | | |
| 20 mM Histidine, 150 mM NaCl pH 5.6 | 1 | 2-8 C. | No Particulates |
| with 0.1% Poloxamer 188 | | | |
| 20 mM Histidine, 150 mM NaCl pH 5.8 | 1 | 2-8 C. | No Particulates |
| with 0.1% Poloxamer 188 | | | |
| 20 mM Succinate, 150 mM NaCl, 5.7 | 1 | 2-8 C. | No Particulates |
| with 0.1% Poloxamer 188 | | | |
| 20 mM Succinate, 150 mM NaCl, 6.0 | 1 | 2-8 C. | No Particulates |
| with 0.1% Poloxamer 188 | | | |
| 20 mM HEPES, 150 mM NaCl, pH 7.0 | 1 | 2-8 C. | No Particulates |
| with 0.1% Poloxamer 188 | | | |
| 20 mM Tris-HCl, 150 mM NaCl, pH 8.0 | 1 | 2-8 C. | No Particulates |
| with 0.1% Poloxamer 188 | | | |

### CONCLUSIONS

The various buffer and pH conditions tested had no detrimental effect on the ability of Poloxamer 188 to inhibit agitation-induced aggregation.

### Example 10

### Effect of Poloxamer of Varying Molecular Weight Ranges on Agitation-Induced Aggregation

The stability of the 15-valent Pneumococcal Conjugate Vaccine-15 (PCV-15) was studied with various commercially available Poloxamers after rotational agitation studies. PCV-15 was prepared in 20 mM histidine pH 5.8 and 150 mM sodium chloride with 0.25 mg/mL Aluminum Phosphate Adjuvant (APA). The CRM₁₉₇ protein at 64 μg/mL conjugated to Pneumococcal polysaccharide (PnP) Types 1, 3, 4, 5, 6A, 7F, 9V, 14, 18C, 19A, 19F, 22F, 23F, and 33F at 4 μg/mL and Type 6B at 8 μg/mL, for a total polysaccharide concentration of 64 μg/mL.

For the rotational agitation studies, the PCV-15 formulation backbone was prepared with the addition of Poloxamer 237, Poloxamer 338, or Poloxamer 407 at the specified concentrations. The agitation studies were designed using rotational side agitation for 24 hours at 4° C. Aggregation was observed visually.

Results:

PCV15 Formulations after rotational agitation in vials for 24 hrs at 4° C. with 0.1% Poloxamer 237 resulted in no observed particulates. The other conditions tested resulted in very small particulates, but were a significant improvement over control formulations without poloxamers.

TABLE 9
Impact of Poloxamers of Varying Molecular Weight
Ranges on Agitation Induced Aggregation formation
in Vials of PCV15 with Aluminum

| | |
|---|---|
| 20 mM Histidine, 150 mM NaCl, 0.05% | Very small participates, |
| Poloxamer 237 pH 5.8 | aggregates |
| 20 mM Histidine, 150 mM NaCl, 0.1% | No Particulates |
| Poloxamer 237 pH 5.8 | |
| 20 mM Histidine, 150 mM NaCl, 0.05% | Very small particulates, |
| Poloxamer 338 pH 5.8 | aggregates |
| 20 mM Histidine, 150 mM NaCl, 0.1% | Very small particulates, |
| Poloxamer 338 pH 5.8 | aggregates |
| 20 mM Histidine, 150 mM NaCl, 0.005% | Very small participates, |
| Poloxamer 407 pH 5.8 | aggregates |
| 20 mM Histidine, 150 mM NaCl, 0.01% | Very small particulates, |
| Poloxamer 407 pH 5.8 | aggregates |
| 20 mM Histidine, 150 mM NaCl, 0.05% | Very small particulates, |
| Poloxamer 407 pH 5.8 | aggregates |
| 20 mM Histidine, 150 mM NaCl, 0.1% | Very small participates, |
| Poloxamer 407 pH 5.8 | aggregates |

Static light scattering was also performed on the sample with Poloxamer 237 at 0.1% to look at size distributions. This formulation was comparable to the population seen in the non-agitated samples (FIG. 7).

### CONCLUSIONS

Poloxamers of varying molecular weight ranges differed in their ability to inhibit agitation-induced aggregation. Optimization of conditions should allow complete inhibition.

1. A formulation comprising (i) a pH buffered saline solution having a pH in the range from 5.0 to 8.0, (ii) a poloxamer having a molecular weight in the range from 1100 to 17,400 and (iii) one or more polysaccharide-protein conjugates.

2. The poloxamer of claim 1 having a molecular weight in the range from 7,500 to 15,000.

3. The poloxamer of claim 1 having a molecular weight in the range from 7,500 to 10,000.

4. The formulation of claim 1, wherein the poloxamer is poloxamer 188 or poloxamer 237.

5. The formulation of claim 1, wherein the final concentration of the poloxamer in the formulation is from 0.001% to 5% weight/volume of the formulation.

6. The formulation of claim 1, wherein the final concentration of the poloxamer in the formulation is from 0.025% to 1% weight/volume of the formulation.

7. The formulation of claim 1, wherein the final concentration of poloxamer 188 in the formulation is from 0.05% to 1.0% weight/volume of the formulation or the final concentration of poloxamer 237 in the formulation is from 0.1% to 1.0% weight/volume of the formulation.

8. The formulation of claim 1, wherein the pH buffered saline solution has a pH in the range from 5.2 to 8.0.

9. The formulation of claim 1, wherein the buffer selected from the group consisting of Tris, phosphate, succinate, histidine, MES, MOPS, HEPES, acetate or citrate.

10. The formulation of claim 1, wherein the buffer is histidine at a final concentration of 5 mM to 50 mM, or succinate at a final concentration of 1 mM to 20 mM.

11. The formulation of claim 1, wherein the saline is present at a concentration from 20 nM to 170 nM.

12. The formulation of claim 1, wherein the protein is the carrier protein CRM₁₉₇.

13. The formulation of claim 1, wherein the polysaccharide-protein conjugate comprises one or more pneumococcal polysaccharides.

14. The formulation of claim 1, wherein the polysaccharide-protein conjugate formulation is a 15-valent pneumococcal conjugate (15vPnC) formulation comprising a S. pneumoniae serotype 1 polysaccharide conjugated to a CRM₁₉₇ polypeptide, a S. pneumoniae serotype 3 polysaccharide conjugated to a CRM₁₉₇ polypeptide, a S. pneumoniae serotype 4 polysaccharide conjugated to a CRM₁₉₇ polypeptide, a S. pneumoniae serotype 5 polysaccharide conjugated to a CRM₁₉₇ polypeptide, a S. pneumoniae serotype 6A polysaccharide conjugated to a CRM₁₉₇ polypeptide, a S. pneumoniae serotype 6B polysaccharide conjugated to a CRM₁₉₇ polypeptide, a S. pneumoniae serotype 7F polysaccharide conjugated to a CRM₁₉₇ polypeptide, a S. pneumoniae serotype 9V polysaccharide conjugated to a CRM₁₉₇ polypeptide, a S. pneumoniae serotype 14 polysaccharide conjugated to a CRM₁₉₇ polypeptide, a S. pneumoniae serotype 18C polysaccharide conjugated to a CRM₁₉₇ polypeptide, a S. pneumoniae serotype 19A polysaccharide conjugated to a CRM₁₉₇ polypeptide, a S. pneumoniae serotype 19F polysaccharide conjugated to a CRM₁₉₇ polypeptide, a S. pneumoniae serotype 22F polysaccharide conjugated to a CRM₁₉₇ polypeptide, a S. pneumoniae serotype 23F polysaccharide conjugated to a CRM₁₉₇ polypeptide, and a S. pneumoniae serotype 33F polysaccharide conjugated to a CRM₁₉₇ polypeptide.

15. The formulation of claim 1, wherein the formulation further comprises an adjuvant.

16. The immunogenic composition of claim 15, wherein the adjuvant is an aluminum-based adjuvant.

17. The formulation of claim 16, wherein the adjuvant is aluminum phosphate.

18. The formulation of claim 17 comprising 0.112 to 0.130 mg elemental aluminum, 140 to 160 mM sodium chloride and 18 to 22 mM L-histidine buffer.

19. The formulation of claim 18, which is a single 0.5 mL dose formulated to contain: 1.8 to 2.2 μg of each saccharide, except for 6B at 3.6 to 4.4 μg; about 32 μg CRM₁₉₇ carrier protein; 0.125 mg of elemental aluminum (0.5 mg aluminum phosphate) adjuvant; about 150 mM sodium chloride and about 20 mM L-histidine buffer.

20. The formulation of claim 1, wherein the formulation further comprises a preservative which is m-cresol, phenol, 2-phenoxyethanol, chlorobutanol, benzyl alcohol, or thimerosal.

21. A vial or pre-filled syringe comprising the formulation of claim 1.
